(19) United States
(12) Patent Application Publication
(10) Pub. No.: US 2004/0092528 A1
(43) Pub. Date: May 13, 2004

(54) Agent for modulating excitatory synaptic transmission comprising a compound having alpha7 nicotinic acetylcholine receptor activation property

(76) Inventors: John S Kelly, Scotland (GB); Joseph Hodgkiss, Scotland (GB); John Sharkey, Scotland (GB)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/362,688

(22) PCT Filed: Sep. 5, 2001
(86) PCT No.: PCT/GB01/03992

(30) Foreign Application Priority Data
Sep. 6, 2000 (GB) 00218859

(51) Int. Cl. A61K 31/495; A61K 31/16; A61K 31/18

(52) U.S. Cl. 514/255.01; 514/602; 514/616

(57) ABSTRACT

The present invention relates to an agent for modulating excitatory synaptic transmission which contains a compound having α7 nicotinic acetylcholine receptor activation property as an active ingredient, and the production of such an agent. It further relates to a pharmaceutical composition which contains such a compound together with a pharmaceutically acceptable carrier or excipient, and a method for modulating excitatory synaptic transmission which comprises administering such a composition. There is also provided a method for screening an agent for modulating excitatory synaptic transmission which comprises using α7 nicotinic acetylcholine receptor activation property as an index. The present invention is useful for the prophylaxis and/or treatment of cerebral diseases, including dementia, amnesia, manic-depressive psychosis, schizophrenia, Parkinson's disease and psychosomatic disease.

The present invention relates to an agent for modulating excitatory synaptic transmission which contains a compound having α7 nicotinic acetylcholine receptor activation property as an active ingredient, and the production of such an agent. The agent is useful for the prophylaxis and/or treatment of cerebral diseases, including dementia and amnesia. The present invention further relates to a pharmaceutical composition which contains a compound having α7 nicotinic acetylcholine receptor activation property together with a pharmaceutically acceptable carrier or excipient, and a method for modulating excitatory synaptic transmission which comprises administering such a composition. The present invention still further relates to a method for screening an agent for modulating excitatory synaptic transmission which comprises using α7 nicotinic acetylcholine receptor activation property as an index.

The area of the brain known as the hippocampus is said to be responsible for learning and memory. Alzheimer's disease (AD) and related dementias are progressive neuro-degenerative diseases of unknown aetiology in which characteristic lesions in the cerebral cortex and hippocampus are associated with cognitive decline and cholinergic neuronal pathologies (Davies, & Maloney, 1976; Bartus et al, 1982; Araujo et al, 1988; Gallagher & Colombo, 1995). Although loss of cholinergic function and the appearance of the characteristic pathology are related to cognitive decline such associations do not signify a causal relationship (Fibiger, 1991; Francis et al, 1999). In AD there is a loss of projecting transmitter systems to the cortex and hippocampus, not only cholinergic but also noradrenergic and serotonergic afferents (Palmer, 1996; Kasa et al, 1997), and at present no cure is in prospect. In the absence of a cure palliative treatment is aimed at up-regulating the function of the reduced pool of available cholinergic neurones. In the brain, as in the periphery, it is possible to enhance synaptic transmission either pre-synaptically, by increasing the amount of transmitter released, or post-synaptically, by modulating the behaviour of the post-synaptic receptors or reducing the breakdown of transmitter. In AD there is a loss of choline acetyltransferase ChAt (see Kasa et al, 1997 for references), thus treatments have concentrated on up-regulating cholinergic transmission in the brain, particularly by inhibition of AchE (Francis et al, 1999) although other therapies are under active consideration (Chorvat et al, 1995; Mucke, 1998; Kelly, 1999). It has also been suggested that activation of somatostatinergic neurones in the hippocampus might provide an indirect means of strengthening cholinergic drive to facilitate cognitive function (Yamazaki, et al, 1996).

As a result of the intensive studies of the present inventors, it has been newly found that a compound having α7 nicotinic acetylcholine receptor activation property has the effect of modulating excitatory synaptic transmission. Based on this new finding, the inventors have found that administration of a compound having α7 nicotinic acetylcholine receptor activation property leads to the prophylaxis and/or treatment of cerebral diseases such as dementia, amnesia, manic-depressive psychosis, schizophrenia, Parkinson's disease, psychosomatic disease, and the like, which resulted in the completion of the present invention.

Accordingly, the present invention provides the following:—

(1) An agent for modulating excitatory synaptic transmission, which comprises a compound having α7 nicotinic acetylcholine receptor activation property as an active ingredient;

(2) A method for modulating excitatory synaptic transmission, comprising administering an effective amount of a compound having α7 nicotinic acetylcholine receptor activation property;

(3) Use of a compound having α7 nicotinic acetylcholine receptor activation property for the production of an agent for modulating excitatory synaptic transmission;

(4) A pharmaceutical composition for modulating excitatory synaptic transmission, which comprises a compound having α7 nicotinic acetylcholine receptor activation property, and a pharmaceutically acceptable carrier or excipient.

(5) The agent, the method and the pharmaceutical composition of any of (1), (2) or (4), which is for the prophylaxis and/or treatment of cerebral diseases; and the use according to (3), which is for the production of an agent for the prophylaxis and/or treatment of cerebral diseases.

(6) The agent, the method and the pharmaceutical composition of (5), which is for the prophylaxis and/or treatment of dementia or amnesia; and the use according to (5), which is for the production of an agent for the prophylaxis and/or treatment of dementia or amnesia.

(7) A method for screening an agent for modulating excitatory synaptic transmission, which comprises using α7 nicotinic acetylcholine receptor activation property as an index.

(8) The screening method of (7), which is a screening method of an anti-dementia agent or anti-amnesia agent.

(9) The agent, the method, the use and the pharmaceutical composition according to (1) to (4), wherein the compound having the α7 nicotinic acetylcholine receptor activation property is obtained by the screening method of (8).

(10) A compound selected by the screening method described in any of (8) or (9).

The compound having α7 nicotinic acetylcholine receptor activation property to be used in the present invention encompasses any compound having such an activation property. Preferable examples thereof include compounds of the following formulae:

\mathring{1} formula [I]

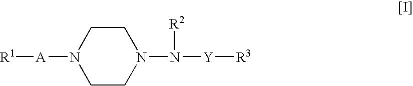

wherein

R¹ is lower alkyl, aryl, ar(lower)alkoxy or heterocyclic group, each of which may be substituted with halogen,

R² is hydrogen atom or lower alkyl,

R³ is cyclo(lower)alkyl, aryl or ar(lower)alkyl, each of which may be substituted with halogen,

A is —CO—, —SO₂— or lower alkylene, and

Y is —CO—, —SO₂— or —CONH—

(EP Publication No. 436734) (hereinafter also referred to as compound [I]), and pharmaceutically acceptable salts thereof and \mathring{2} formula [II-1]:

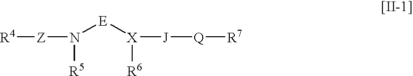

wherein

R⁴ is acyl,

R⁷ is lower alkyl, lower alkoxy, lower alkylamino, lower alkenyl, lower alkenyloxy, lower alkenylamino, lower alkynyl, lower alkynyloxy, lower alkynylamino, cyclo(lower)alkyl, cyclo(lower)alkyloxy, cyclo(lower)alkylamino, aryl, aryloxy, arylamino, a heterocyclic group or amino substituted with a heterocyclic group, each of which may be substituted with suitable substituent(s); or acyl;

Z is a single bond, —CO— or —SO₂—,

E is lower alkylene optionally substituted with suitable substituent(s),

X is CH or N,

J is a single bond, lower alkylene or

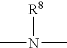

wherein R⁸ is hydrogen, lower alkyl, substituted-lower alkyl, an N-protective group, aryl, acyl or a heterocyclic group,

Q is —CH₂—, —CO—, —SO₂— or —N═CH—, and

R⁵ and R⁶ are each hydrogen or lower alkyl, or are taken together to form lower alkylene optionally condensed with a cyclic hydrocarbon or a heterocyclic ring, provided that when X is N, then

1) J is a single bond, and Q is —CH₂—, —CO— or —SO₂—, or

2) J is lower alkylene,

(hereinafter also referred to as compound [II-1]) and pharmaceutically acceptable salts thereof.

Preferred compound [I] is one which has a lower alkyl, phenyl, naphthyl or thienyl for R¹, hydrogen or lower alkyl for R², phenyl which may be substituted with a halogen for R³, —CO— for A, and —CO— or —SO₂-for Y.

More preferred compound [I] is one which has a lower alkyl for R¹, hydrogen for R², phenyl which is substituted with a halogen for R³, —CO— for A, and —CO— for Y.

Most preferred compound [I] is N-(4-acetyl-1-piperazinyl)-p-fluorobenzamide monohydrate (compound 1) (International Publication No. WO98/25914) and which is designated FK960 and has the formula:

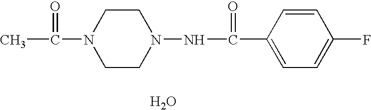

When Z is a single bond, E is ethylene, and R⁵ and R⁶ are taken together to form ethylene, preferable compounds [II-1] can be represented by the following general formula [II-2]:

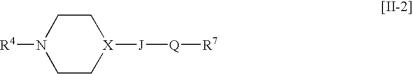

wherein

R⁴ is acyl,

R⁷ is aryl, aryloxy or arylamino, the aryl moiety of all of which may be substituted with halogen; pyridyl; or pyridylamino;

X is CH or N,

J is a single bond, lower alkylene or

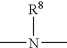

wherein R⁸ is hydrogen, lower alkyl or an N-protective group,

is —CH₂—, —CO— or —SO₂—, provided that when X is N, then J is a single bond or lower alkylene, (hereinafter also referred to as compound [II-2]) and pharmaceutically acceptable salts thereof.

Preferred compound [II-2] is one which has lower alkanoyl, esterified carboxy, substituted or unsubstituted aroyl, lower alkylsulfonyl, substituted or unsubstituted arylsulfonyl, or cyclo(lower)alkylcarbonyl for R⁴, and aryl or arylamino, each aryl of which may be substituted with halogen for R⁷, CH or N for X, a single bond, lower alkylene or

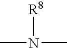

(wherein R⁸ is hydrogen, lower alkyl or an N-protective group) for J, and —CH₂—, —CO— or —SO₂— for Q, provided that when X is N, then J is a single bond or lower alkylene, and pharmaceutically acceptable salts thereof.

More preferred compound [II-2] is one which has lower alkanoyl, lower alkoxycarbonyl, aroyl, aroyl substituted with halo(lower)alkoxy, lower alkylsulfonyl, arylsulfonyl, arylsulfonyl substituted with halogen, or cyclo(lower)alkylcarbonyl for R⁴, aryl or arylamino, each aryl of which may be substituted with halogen for R⁷, CH for X, a single bond or —NH— for J, and —CO— or —SO₂— for Q, and pharmaceutically acceptable salts thereof.

Particularly more preferred compound [II-2] is one which has lower alkanoyl, lower alkoxycarbonyl, aroyl, aroyl substituted with halo(lower)alkoxy, lower alkylsulfonyl, arylsulfonyl, arylsulfonyl substituted with halogen, or cyclo(lower)alkylcarbonyl for R⁴, aryl or arylamino, each aryl of which may be substituted with halogen for R⁷, CH for X, —NH— for J, and —CO— for Q, and pharmaceutically acceptable salts thereof.

Most preferred compound [II-2] is one selected from the group consisting of N-(1-acetylpiperidin-4-yl)-4-fluorobenzamide, N-(1-acetylpiperidin-4-yl)-N′-(4-fluorophenyl)urea, 4-(4-fluorobenzoylamino)-1-methoxycarbonylpiperidine, 4-(4-fluorobenzoylamino)-1-(4-fluorophenylsulfonyl)piperidine, 4-(4-fluorobenzoylamino)-1-(4-trifluoromethoxybenzoyl)piperidine, 4-(4-fluorobenzoylamino)-1-methylsulfonylpiperidine, N-(1-methoxycarbonylpiperidine-4-yl)-N′-(4-fluorophenyl)urea, N-(1-(4-fluorophenylsulfonyl)piperidin-4-yl)-N′-(4-fluorophenyl)-urea, N-(1-benzoylpiperidin-4-yl)-4-fluorobenzamide, N-(1-pivaloylpiperidin-4-yl)-4-fluorobenzamide and N-(1-cyclopropylcarbonylpiperidin-4-yl)-4-flurobenzamide.

In the above and subsequent description of the present specification, suitable examples of the various definitions to be included within the scope of the invention are explained in detail in the following.

The term “lower” is intended to mean a group having 1 to 6 carbon atom(s), unless otherwise provided.

The lower moiety in the terms “lower alkenyl”, “lower alkenyloxy”, “lower alkenylamino”, “lower alkynyl”, “lower alkynyloxy” and “lower alkynylamino” is intended to mean a group having 2 to 6 carbon atoms.

The lower moiety in the terms “cyclo(lower)alkyl”, “cyclo(lower)alkyloxy” and “cyclo(lower)alkylamino” is intended to mean a group having 3 to 6 carbon atoms.

Suitable “lower alkyl” and lower alkyl moiety in the terms “substituted-lower alkyl”, “ar(lower)alkyl”, “halo(lower)alkyl”, “lower alkylamino”, “lower alkylsilyl”, “lower alkylthio” and “lower alkylsulfonyl” may be a straight or branched C₁-C₆ alkyl such as methyl, ethyl, propyl, isopropyl, butyl, isobutyl, tert-butyl, pentyl, ethylpropyl, hexyl or the like, in which preferable on is methyl.

Suitable “lower alkenyl” and lower alkenyl moiety in the terms “lower alkenyloxy” and “lower alkenylamino” may be a straight or branched C₂-C₆ alkenyl such as ethenyl, propenyl, butenyl, pentenyl, hexenyl, isopropenyl, butadienyl, pentadienyl, hexadienyl or the like, in which preferable one is ethenyl, propentyl or butadienyl.

Suitable “lower alkynyl” and lower alkynyl moiety in the terms “lower alkynyloxy” and “lower alkynylamino” may be a straight or branched C₂-C₆ alkynyl such as ethynyl, propargyl, butynyl or the like, in which preferable one is ethynyl.

Suitable “cyclo(lower)alkyl” and cyclo(lower)alkyl moiety in the terms “cyclo(lower)alkyloxy” and “cyclo(lower)alkylamino” may be cyclo(C₃-C₆)alkyl such as cyclopropyl, cyclobutyl, cyclopentyl or cyclohexyl, in which preferable one in the definitions of general formulas [II-1] and [II-2]is cyclopropyl.

Suitable “aryl” in the definitions of general formula [I] may be phenyl, naphthyl, tolyl, xylyl, mesityl, cumenyl, and the like, in which preferable one is phenyl or naphthyl.

Suitable “ar(lower)alkoxy” in the definitions of general formula [I] may be benzyloxy, phenethyloxy, phenylpropoxy, benzhydryloxy, trityloxy and the like.

Suitable “aryl” and aryl or ar moiety in the terms “ar(lower)alkoxy”, “aryloxy”, “arylamino”, “arylsulfonyl”, “aroyl” and “ar(lower)alkyl” in the definitions of general formulas [II-1] and [II-2] may be phenyl, naphthyl, phenyl substituted with lower alkyl [e.g. tolyl, xylyl, mesityl, cumenyl, di(tert-butyl)phenyl, etc.] and the like, in which preferable one is phenyl or tolyl.

Suitable “ar(lower)alkyl” may be benzyl, phenethyl, phenylpropyl, benzhydryl, trityl and the like, in which preferable one in the definitions of general formulas [II-1] and [II-2] is benzyl.

Suitable “lower alkylene” in the definitions of general formula [I] may be methylene, ethylene, propylene, pentamethylene, hexamethylene, and the like.

Suitable “lower alkylene” and lower alkylene moiety in the term “lower alkylenedioxy” in the definitions of general formulas [II-1] and [II-2] may be a straight or branched C₁-C₆ alkylene such as methylene, ethylene, trimethylene, propylene, tetramethylene, pentamethylene, hexamethylene, ethylethylene or the like, in which preferable one is methylene, ethylene or trimethylene.

Suitable “lower alkoxy” and lower alkoxy moiety in the terms “ar(lower)alkoxy” and “halo(lower)alkoxy” may be a straight or branched C₁-C₆ alkoxy such as methoxy, ethoxy, propoxy, isopropoxy, methylpropoxy, butoxy, isobutoxy, tert-butoxy, pentyloxy, hexyloxy or the like, in which preferable one is methoxy or tert-butoxy.

Suitable “heterocyclic group” in the definitions of general formula [I] may include saturated or unsaturated, monocyclic or polycyclic one containing at least one hetero atom such as nitrogen atom, oxygen atom or sulfur atom. The preferred examples of thus defined “heterocyclic group” may be unsaturated, 3 to 8-membered, more preferably 5 or 6-memebered heteromonocyclic group containing 1 to 4-nitrogen atom(s), for example, pyrrolyl, imidazolyl, pyrazolyl, pyridyl, pyridyl N-oxide, dihydropyridyl, tetrahydropyridyl, pyrimidyl, pyrazinyl, pyridazinyl, triazinyl, triazolyl, tetrazinyl, tetrazolyl, etc.;

unsaturated, condensed heterocyclic group containing 1 to 5 nitrogen atom(s), for example, indolyl, isoindolyl, indolizinyl, benzimidazolyl, quinolyl, isoquinolyl, indazolyl, benzotriazolyl, etc.;

unsaturated, 3 to 8-membered heteromonocyclic group containing 1 to 2 oxygen atom(s) and 1 to 3 nitrogen atom(s), for example, oxazolyl, isoxazolyl, oxadiazolyl etc.;

saturated, 3 to 8-membered heteromonocyclic group containing 1 to 2 oxygen atom(s) and 1 to 3 nitrogen atom(s), for example, morpholino, sydnonyl, etc.;

unsaturated, condensed heterocyclic group containing 1 to 2 oxygen atom(s) and 1 to 3 nitrogen atom(s), for example, benzoxazolyl, benzoxadiazolyl, etc.;

unsaturated, 3 to 8-membered heteromonocyclic group containing 1 to 2 sulfur atom(s) and 1 to 3 nitrogen atom(s), for example, thiazolyl, isothiazolyl, thiadiazolyl etc.;

unsaturated, 3 to 8-membered heteromonocyclic group containing 1 to 2 sulfur atom(s), for example, thienyl, etc.;

unsaturated, condensed heterocyclic group containing 1 to 2 sulfur atom(s) and 1 to 3 nitrogen atom(s), for example, benzothiazolyl, benzothiadiazolyl, etc.;

unsaturated, 3 to 8-membered heteromonocyclic group containing an oxygen atom, for example, furyl, etc.;

unsaturated, condensed heterocyclic group containing 1 to 2 sulfur atom(s), for example, benzothienyl, etc.;

unsaturated, condensed heterocyclic group containing 1 to 2 oxygen atom(s), for example, benzofuranyl, etc.; or the like.

The above-mentioned “lower alkyl”, “aryl”, “ar(lower)alkoxy”, “heterocyclic group”, “cyclo(lower)alkyl” and “ar(lower)alkyl” in the definitions of general formula [I] may be substituted with halogen [e.g. fluorine, chlorine, bromine and iodine].

Suitable “halogen” and halo moiety in the term “halo(lower)alkyl” may be fluorine, chlorine, bromine and iodine, in which preferable one is fluorine, chlorine or iodine.

Suitable “halo(lower)alkyl” may be lower alkyl substituted with one or more halogens such as chloromethyl, dichloromethyl, fluoromethyl, difluoromethyl, trifluoromethyl, pentachloroethyl or the like, in which preferable one is trifluoromethyl.

Suitable “halo(lower)alkoxy” may be lower alkoxy substituted with one or more halogens such as chloromethoxy, dichloromethoxy, fluoromethoxy, difluoromethoxy, trifluoromethoxy, pentachloromethoxy or the like, in which preferable one is trifluoromethoxy.

Suitable “lower alkylamino” may be mono or di(lower)alkylamino such as methylamino, ethylamino, propylamino, isopropylamino, butylamino, tert-butylamino, isobutylamino, pentylamino, hexylamino, dimethylamino, diethylamino, dipropylamino, dibutylamino, diisopropylamino, dipentylamino, dihexylamino, N-methylethylamino or the like, in which preferable one is dimethylamino.

Suitable “lower alkylsilyl” may be mono, di, or tri(lower)alkylsilyl such as trimethylsilyl, dimethylsilyl, triethylsilyl or the like, in which preferable one is trimethylsilyl.

Suitable “lower alkylenedioxy” may be methylenedioxy, ethylenedioxy and the like, in which preferable one is methylenedioxy.

Suitable “heterocyclic group” in the definitions of general formulas [II-1]and [II-2] may be one containing at least one hetero atom selected from nitrogen, sulfur and oxygen atom, and may include saturated or unsaturated, monocyclic or polycyclic heterocyclic group, and preferable heterocyclic group may be N-containing heterocyclic group such as

unsaturated 3 to 6-membered heteromonocyclic group containing 1 to 4 nitrogen atom(s), for example, pyrrolyl, pyrrolinyl, imidazolyl, pyrazolyl, pyridyl, pyrimidinyl, pyrazinyl, pyridazinyl, triazolyl [e.g. 4H-1,2,4-triazolyl, 1H-1,2,3-triazolyl, 2H-1,2,3-triazolyl, etc.], tetrazolyl [e.g. 1H-tetrazolyl, 2H-tetrazolyl, etc.], etc.;

saturated 3 to 7-membered heteromonocyclic group containing 1 to 4 nitrogen atom(s), [e.g. pyrrolidinyl, imidazolidinyl, piperidyl, piperazinyl, homopiperazinyl, etc.];

unsaturated condensed heterocyclic group containing 1 to 5 nitrogen atom(s), for example, indolyl, isoindolyl, indolizinyl, benzimidazolyl, quinolyl, isoquinolyl, imidazopyridyl, indazolyl, benzotriazolyl, tetrazolopyridazinyl [e.g. tetrazolo[1,5-b]pyridazinyl, etc.], quinoxalinyl, etc.;

unsaturated 3 to 6-membered heteromonocyclic group containing an oxygen atom, for example, pyranyl, furyl, etc.;

saturated 3 to 6-membered heteromonocyclic group containing an oxygen atom, for example, 1H-tetrahydropyranyl, tetrahydrofuranyl, etc.;

unsaturated 3 to 6-membered heteromonocyclic group containing 1 to 2 sulfur atom(s), for example, thienyl, etc.;

unsaturated 3 to 6-membered heteromonocyclic group containing 1 to 2 oxygen atom(s) and 1 to 3 nitrogen atom(s), for example, oxazolyl, isoxazolyl, oxadiazolyl [e.g. 1,2,4-oxadiazolyl, 1,3,4-oxadiazolyl, 1,2,5-oxadiazolyl, etc.], oxazolinyl [e.g. 2-oxazolyinyl, etc.], etc.;

saturated 3 to 6-membered heteromonocyclic group containing 1 to 2 oxygen atom(s) and 1 to 3 nitrogen atom(s) [e.g. morpholinyl, etc.];

unsaturated condensed heterocyclic group containing 1 to 2 oxygen atom(s) and 1 to 3 nitrogen atom(s) [e.g. benzofurazanyl, benzoxazolyl, benzoxadiazolyl, etc.];

unsaturated 3 to 6-membered heteromonocyclic group containing 1 to 2 sulfur atom(s) and 1 to 3 nitrogen atom(s), for example, thiazolyl, thiadiazolyl [e.g. 1,2,4-thiadiazolyl, 1,3,4-thiadiazolyl, 1,2,5-thiadiazolyl, etc.], etc.;

saturated 3 to 6-membered heteromonocyclic group containing 1 to 2 sulfur atom(s) and 1 to 3 nitrogen atom(s) [e.g. thiazolidinyl, etc.];

unsaturated condensed heterocyclic group containing 1 to 2 sulfur atom(s) and 1 to 3 nitrogen atom(s) [e.g. benzothiazolyl, benzothiadiazolyl, etc.];

unsaturated condensed heterocyclic group containing 1 to 2 oxygen atom(s) [e.g. benzofuranyl, benzodioxolyl, chromanyl, etc.] and the like.

Said “heterocyclic group” may be substituted with lower alkyl as exemplified above, in which preferable one is thienyl, pyridyl, methylpyridyl, quinolyl, indolyl, quinoxalinyl, benzofuranyl or tetramethylchromanyl, and more preferable one is pyridyl.

Suitable “acyl” may be carboxy; esterified carboxy; carbamoyl substituted with lower alkyl, aryl, ar(lower)alky, arylsulfonyl, lower alkylsulfonyl or a heterocyclic group; substituted or unsubstituted arylsulfonyl;lower alkylsulfonyl; cyclo(lower)alkylcarbonyl;lower alkanoyl; substituted or unsubstituted aroyl; a heterocycliccarbonyl and the like.

The esterified carboxy may be substituted or unsubstituted lower alkoxycarbonyl [e.g. methoxycarbonyl, ethoxycarbonyl, propoxycarbonyl, butoxycarbonyl, tert-butoxycarbonyl, hexyloxycarbonyl, 2-iodoethoxycarbonyl, 2,2,2-trichloroethoxycarbonyl, etc.], substituted or unsubstituted aryloxycarbonyl [e.g. phenoxycarbonyl, 4-nitorophenoxycarbonyl, 2-naphthyloxycarbonyl, etc.], substituted or unsubstituted ar(lower)alkoxycarbonyl [e.g. benzyloxycarbonyl, phenethyloxycarbonyl, benzhydryloxycarbonyl, 4-nitrobenzyloxycarbonyl, etc.], and the like, in which preferable one is unsubstituted lower alkoxycarbonyl and more preferable one is methoxycarbonyl or tert-butoxycarbonyl.

The carbamoyl substituted with lower alkyl may be methylcarbamoyl, ethycarbamoyl, propylcarbamoyl, dimethylcarbamoyl, diethylcarbamoyl, N-methyl-N-ethylcarbamoyl and the like.

The carbamoyl substituted with aryl may be phenylcarbamoyl, naphthylcarbamoyl, lower alkyl-substituted phenylcarbamoyl [e.g. tolylcarbamoyl, xylylcarbamoyl, etc.] and the like.

The carbamoyl substituted with ar(lower)alkyl may be benzylcarbamoyl, phenethylcarbamoyl, phenylpropylcarbamoyl and the like, in which preferable one is benzylcarbamoyl.

The carbamoyl substituted with arylsulfonyl may be phenylsulfonylcarbamoyl, tolylsulfonylcarbamoyl and the like.

The carbamoyl substituted with lower alkylsulfonyl may be methylsulfonylcarbamoyl, ethylsulfonylcarbamoyl and the like.

The carbamoyl substituted with a heterocyclic group may be one substituted with a heterocyclic group as mentioned above for the definitions of general formulas [II-1]and [II-2].

The lower alkanoyl may be formyl, acetyl, propionyl, butyryl, isobutyryl, valeryl, isovaleryl, pivaloyl, hexanoyl and the like, in which preferable one is acetyl or pivaloyl.

The substituted or unsubstituted aroyl may be benzoyl, naphthoyl, toluoyl, di(tert-butyl)benzoyl, halo(lower)alkoxybenzoyl [e.g. trifluoromethoxybenzoyl, etc.] and the like, in which preferable one is benzoyl or trifluoromethoxybenzoyl.

The substituted or unsubstituted arylsulfonyl may be phenylsulfonyl, tolylsulfonyl, halophenylsulfonyl [e.g. fluorophenylsulfonyl, etc.] and the like, in which preferable one is fluorophenylsulfonyl.

The lower alkylsulfonyl may be methylsulfonyl, ethylsulfonyl and the like, in which preferable one is methylsulfonyl.

The cyclo(lower)alkylcarbonyl may be cyclo(C₃-C₆)alkylcarbonyl such as cyclopropylcarbonyl, cyclobutylcarbonyl, cyclopentylcarbonyl or cyclohexylcarbonyl, in which preferable one is cyclopropylcarbonyl.

The heterocyclic moiety in the term “a heterocycliccarbonyl” may be one mentioned above as a heterocyclic group for the definitions of general formulas [II-1]and [II-2].

Suitable “N-protective group” may be common N-protective group such as substituted or unsubstituted lower alkanoyl [e.g. formyl, acetyl, propionyl, trifluoroacetyl, etc.], lower alkoxycarbonyl [e.g. tert-butoxycarbonyl, tert-amyloxycarbonyl, etc.], substituted or unsubstituted aralkyloxycarbonyl [e.g. benzyloxycarbonyl, p-nitrobenzyloxycarbonyl, etc.], 9-fluorenylmethoxycarbonyl, substituted or unsubstituted arenesulfonyl [e.g. benzenesulfonyl, tosyl, etc.], nitrophenylsulfeny, aralkyl [e.g. trityl, benzyl, etc.] or the like, in which preferable one is lower alkoxycarbonyl and more preferable one is tert-butoxycarbonyl.

Suitable “cyclic hydrocarbon” may be a saturated or unsaturated cyclic hydrocarbon such as cyclopentane, cyclohexane, benzene, naphthalene, indan, indene or the like.

Suitable “substituted-lower alkyl” may be lower alkyl substituted with halogen, aryl, acyl, lower alkoxy, aryloxy and the like, in which preferable one is benzyl.

Suitable “heterocyclic ring” may be one which is a heterocyclic group, as mentioned above for the definitions of general formulas [II-1]and [II-2], added by hydrogen.

Suitable lower alkylene condensed with a cyclic hydrocarbon may be lower alkylene condensed with benzene and the like, in which preferable one is ethylene condensed with benzene.

Suitable lower alkylene condensed with a heterocyclic ring may be lower alkylene condensed with pyridine and the like, in which preferable one is ethylene condensed with pyridine.

Preferred “acyl” for R⁴ may be lower alkanoyl; lower alkoxycarbonyl; aroyl optionally substituted with halo(lower)alkoxy; arylsulfonyl optionally substituted with halogen; lower alkylsulfonyl; or cyclo(lower)alkylcarbonyl, in which more preferable one is acetyl, pivaloyl, methoxycarbonyl, tert-butoxycarbonyl, benzoyl, trifluoromethoxybenzoyl, fluorophenylsulfonyl, methylsulfonyl or cyclopropylcarbonyl.

Preferred “suitable substituent” as the substituent of lower alkyl, lower alkoxy, lower alkylamino, lower alkenyl, lower alkenyloxy, lower alkenylamino, lower alkynyl, lower alkynyloxy, lower alkynylamino, cyclo(lower)alkyl, cyclo(lower)alkyloxy, cyclo(lower)alkylamino, aryl, aryloxy, arylamino, a heterocyclic group or amino substituted with a heterocyclic group for R⁷ may be halo(lower)alkyl, halo(lower)alkoxy, lower alkenyl, lower alkynyl, lower alkylamino, acylamino, acyl, lower alkylsilyl, lower alkoxy, aryl, lower alkylenedioxy, acyloxy, hydroxy, nitro, amino, cyano, halogen, aryloxy, lower alkylthio and the like.

Preferred “aryl which may be substituted with suitable substituent(s)” for R⁷ may be aryl optionally substituted with halogen, in which more preferable one is fluorophenyl.

Preferred “arylamino which may be substituted with suitable substituent(s)” for R⁷ may be arylamino optionally substituted with halogen, in which preferable one is phenylamino or fluorophenylamino.

Preferred “aryloxy which may be substituted with suitable substituent(s)” for R⁷ may be aryloxy optionally substituted with halogen, in which preferable one is fluorophenoxy.

Preferred “lower alkylene” for J may be methylene.

Preferred “lower alkyl” for R⁸ in J may be methyl.

Preferred “N-protective group” for R⁸ in J may be tert-butoxycarbonyl.

Preferred “suitable substituent” as the substituent of lower alkylene for E may be oxo, lower alkyl, hydroxy(lower)alkyl or acyl, in which more preferable one is oxo, dioxo, methyl, dimethyl, hydroxymethyl, or benzylcarbamoyl.

Preferred “lower alkylene” for E may be methylene, ethylene or trimethylene, and more preferable one is ethylene.

Preferred “lower alkyl” for R⁵ and R⁶ may be methyl.

Preferred “lower alkylene which R⁵ and R⁶ are taken together to form” may be ethylene or trimethylene.

Preferred “a cyclic hydrocarbon with which lower alkylene is condensed” may be benzene.

Another more preferred compound [II-2] is one having lower alkanoyl, lower alkoxycarbonyl, aroyl, aroyl substituted with halo(lower)alkoxy, lower alkylsulfonyl, arylsulfonyl, arylsulfonyl substituted with halogen, or cyclo(lower)alkylcarbonyl for R⁴, aryl or arylamino, each aryl of which may be substituted with halogen for R⁷, N for X, a single bond for J, and —CO— for Q.

Another most preferred compound [II-2] is one selected from the group consisting of

1-acetyl-4-(4-flurophenylcarbamoyl)piperazine,

1-tert-butoxycarbonyl-4-(4-fluorophenylcarbamoyl)piperazine,

1-(4-fluorophenylcarbamoyl)-4-(4-trifluoromethoxybenzoyl)-piperazine and

1-methoxycarbonyl-4-(4-fluorophenylcarbamoyl)piperazine.

Suitable pharmaceutically acceptable salts of the compounds of general formulae [I], [II-1] and [II-2] are conventional non-toxic salts and include acid addition salt such as an inorganic acid addition salt [e.g. hydrochloride, hydrobromide, sulfate, phosphate, etc.], an organic acid addition salt [e.g. formate, acetate, trifluoroacetate, maleate, tartrate, methanesulfonate, benzenesulfonate, toluenesulfonate, etc.], a salt with an amino acid [e.g. aspartic acid salt, glutamic acid salt, etc.], a metal salt such as an alkali metal salt [e.g. sodium salt, potassium salt, etc.] and alkaline earth metal salt [e.g. calcium salt, magnesium salt, etc.] and the like.

Compounds of the formula [I] and salts thereof can be prepared according to the method disclosed in EP Publication No. 436734.

Compounds of the formula [II-1] including compounds [II-2] and salts thereof can be prepared according to the method disclosed in International Publication No. WO 00/42011.

It is to be noted that the compounds [I], [II-1] and [II-2] may include one or more-stereoisomer(s) such as optical isomer(s) or geometrical isomer(s) due to asymmetric carbon atom(s) and double bond(s), and all of such isomers and mixtures thereof are included within the scope of this invention.

Additionally, it is to be noted that any solvate [e.g. enclosure compound (e.g. hydrate, etc.)] of the compound [I], [II-1] and [11-2] and pharmaceutically acceptable salts thereof is also included within the scope of this invention.

The test compound to be subject to the screening method of the present invention is free of any particular limitation and may be selected from natural product, chemically synthesized compound, nucleic acid, peptide, antibody and the like obtained by genetic engineering and their libraries. The test compound is preferably a pure substance, but may be a mixture or racemic compound. The test compound may be also modified to label with radioisotope or may contain modification made during construction of the library. The obtained test compound can be optimized by chemical synthetic method and the like.

By selecting the test compounds using the screening method of the present invention, an agent for modulating excitatory synaptic transmission, particularly an anti-dementia agent, an anti-amnesia agent and the like can be screened.

The test compound selected by the screening method of the present invention and a compound obtained by optimizing this compound are all encompassed in the scope of the present invention.

The compound of the present invention having α7 nicotinic acetylcholine receptor activation property can be used in the dosage form of a solid, semi-solid or liquid preparation in conjunction with an organic or inorganic carrier or excipient, which is suitable for rectal administration, pulmonary (pernasal or buckle inhalation), nasal drop, eye drop, external (local), oral or parenteral (subcutaneous, intravenous or intramuscular) administration and the like, direct administration to diseased region, such as brain, spinal fluid, cerebroventricle and the like, or inhalation.

A compound having α7 nicotinic acetylcholine receptor activation property can be admixed with a pharmaceutically acceptable substantially non-toxic carrier or excipient conventionally used for dosage forms suitable for use, such as tablets, pellets, troches, capsules, suppositories, cream, ointment, aerosol, inhalable powder medicine, liquid, emulsion, suspension, and the like. Where necessary, auxiliary, stabilizer, tackifier, coloring agent and flavor can be used.

The agent for modulating excitatory synaptic transmission, particularly an anti-dementia agent and an anti-amnesia agent, of the present invention can be produced by a method conventionally used in the pertinent field. Where necessary, a method routinely used in this technical field can be used for the production of these drugs for an improved bioavailability.

The agent for modulating excitatory synaptic transmission, particularly an anti-dementia agent and an anti-amnesia agent, of the present invention is preferably administered intravenously (inclusive of addition into infusion), intramuscularly or orally when applying to humans or animals.

The agent for modulating excitatory synaptic transmission, particularly an anti-dementia agent and an anti-amnesia agent, of the present invention is contained in a preparation in an amount sufficient to provide a desired prophylactic and/or treatment effect on the progression and conditions of diseases.

The amount and administration route of the compound having α7 nicotinic acetylcholine receptor activation property are subject to variation depending on the kind of compound, age and conditions of the patients to be the subject of the prophylaxis and/or treatment. When compound 1 is used, for example, the daily dose is 0.01-10 mg/kg body weight by oral administration, which is given once to several times a day for the treatment and/or prophylaxis of the aforementioned diseases.

The present invention will now be described in more detail by reference to studies conducted by the present inventors on the effect of the aforementioned compound FK960 on glutamatergic transmission in CA1 neurones in rat hippocampal slices. FK960 has previously been shown to reverse scopolamine-induced cognitive deficits in rats in vivo (Yamazaki et al, 1996) and to increase the magnitude of long-term potentiation in guinea-pig hippocampus in vitro (Matsuoka & Satoh, 1998). More recently it has been shown that FK960 increases the amplitude of the un-potentiated population spike in rat hippocampus (Matsuyama, et al, 2000). These studies implicated somatostatinergic, cholinergic and serotonergic systems but did not, however, indicate whether FK960 acts pre- or post-synaptically nor did they rule out the involvement of other transmitter systems, such as glutamate or GABA. A role for glutamatergic transmission in memory has been advanced by studies with AMPAkines such as CX516 (1-(quinoxalin-6-ylcarbonyl)piperidine, also listed as BDP-12, Cortex Pharmaceuticals) and related compounds which enhanced memory (Granger et al, 1996; Lynch et al, 1996), increased the degree and duration of long-term potentiation (Staubli et al, 1994) and the amplitude and duration of the field EPSP in the hippocampus by changing the characteristics of AMPA-receptor desensitisation and/or de-activation (Arai et al 1996a,b; Sirvio et al 1996, Arai & Lynch, 1998). The work of the present inventors shows FK960 to enhance transmitter release at AMPAergic synapses on CA1 neurones in the hippocampus and raises the possibility that FK960 acts on the nerve terminal to increase transmitter release. Given the recent work which has shown nicotine to have a similar action the effect of block of the α7-subtype of the nicotinic receptor by methyllycaconitine or α-bungarotoxin in the action of FK960 was also examined to determine the involvement, if any, of this sub-type of ACh receptor.

The present invention will now be further illustrated by reference to the following Examples and the accompanying drawings in which:—

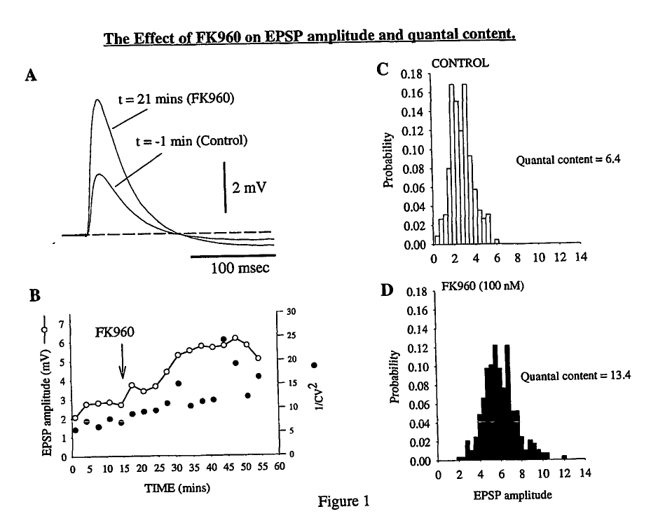

FIG. 1 shows the effect of FK960 on EPSP amplitude and quantal content. Averaged records of 50 consecutive excitatory post-synaptic potentials (EPSPs) recorded from a hippocampal CA1 neurone 1 min 40 s before and 21 min 40 sec after exposure to FK960 are shown (A), the slope of the rising phase of the averaged EPSP increased from 326.7 mV/s to 742.8 mV/s in this experiment, an increase of 127%. There was a corresponding increase in mean EPSP amplitude (open circles) and 1/CV² (a measure of quantal content, filled circles), the time courses of which are shown in (B); FK960 was added at t=15 mins, each point was determined from 50 consecutive EPSPs. Amplitude histograms of EPSPs before (C) and after (D) exposure to 100 nM FK960. Mean EPSP amplitude was 2.6 mV±1.0 (SD, n=225) in control aCSF and it increased by 123% to 5.8 mV±1.6 (SD, n=287) in FK960, indicated by the rightward shift of the distribution. After correction for the variance of the baseline noise the quantal content increased from 6.4 to 13.4, a rise of 109%.

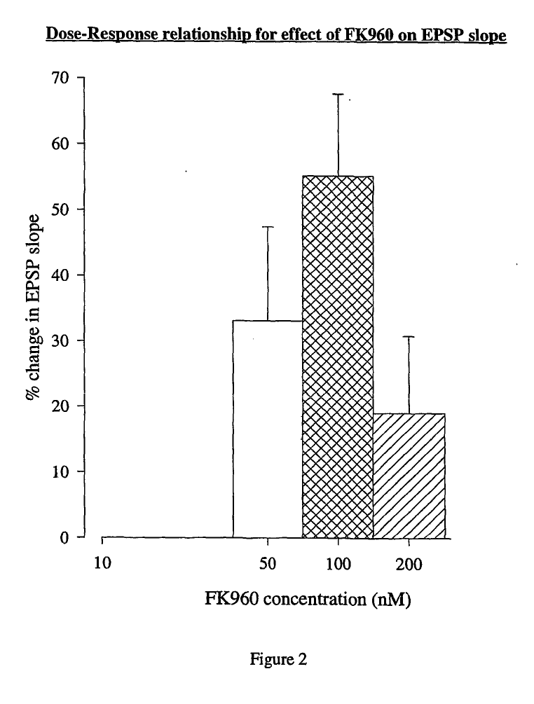

FIG. 2 shows the dose response relationship for the effect of FK960 on EPSP slope. The graph is based on data from 21 neurones. In the absence of FK960 an 11%±10 (SEM, n=6) increase in slope was seen after 21 mins. This contrasted with an increase of 55%±13 (n=5) seen after exposure to 100 nM FK960 which was significantly greater than control (P=0.04, one-way ANOVA). The increases in EPSP slope seen with 50 nM and 200 nM were smaller and not significant.

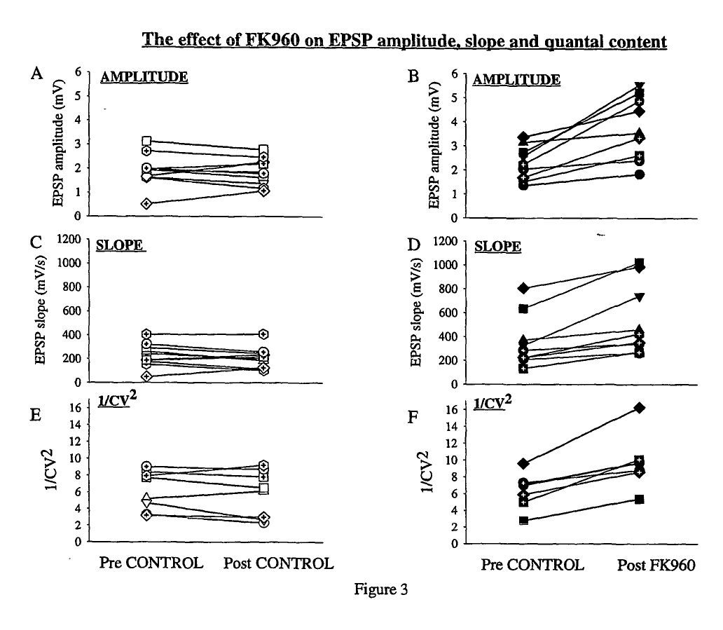

FIG. 3 shows the effect of FK960 on EPSP amplitude, slope and quantal content. EPSP amplitudes (A, B), slopes (C, D) and quantal content (1/CV², the reciprocal of the square of the coefficient of variation, E, F) were determined before (pre-control) and 21 min 40 sec after exposure to either FK960 (post-FK960, n=9 neurones) or control aCSF (post-control, n=10 neurones). EPSP amplitudes were measured individually and the mean of 50 consecutive EPSPs before and after changeover to either control aCSF (A) or FK960 (B) was determined. The slopes were determined by averaging 50 consecutive EPSPs at the appropriate times and calculating the slope of the rising phase of the averaged EPSP between 10% and 50% of peak amplitude. The increases in EPSP amplitude and slope were significantly greater after treatment with FK960. The value 1/CV² (a measure of the mean number of quanta released) was determined from 100 EPSPs recorded prior to solution changeover (pre-control) and compared to that determined from the same number of EPSPs recorded after 20 min exposure to either control aCSF (E) or FK960 (F). The neurones exposed to control aCSF showed a mean decrease of 6.5% in 1/CV² (E), whereas a mean increase of 53% was seen in the group exposed FK960 (F).μ

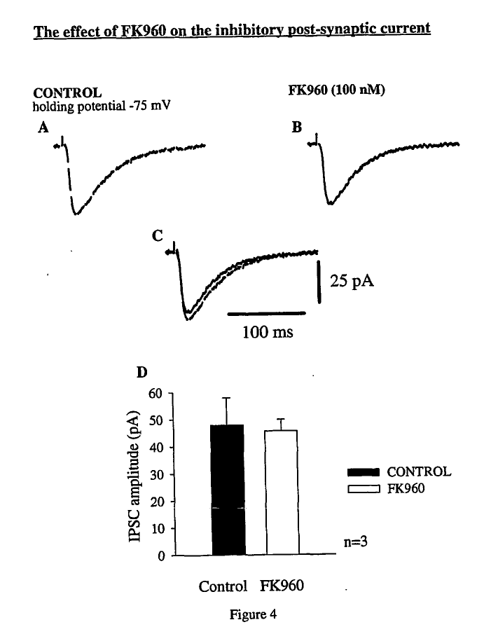

FIG. 4 shows the effect of FK960 on the inhibitory post-synaptic current (IPSC). 100 nM FK960 had no significant effect on the IPSC in CA1 neurones. Hippocampal slices were exposed to an aCSF (control aCSF) containing CNQX (15 μM) and D-AP5 (50 μM) to block AMPA and NMDA receptors respectively. Stimulation of the stratum radiatum at 0.1 Hz in the presence of these antagonists elicited a response consisting of GABA_{A} and GABA_{B} components. Averaged records of 15 IPSCs recorded from a CA1 neurone voltage clamped at −75 mV in control aCSF are shown in A; the IPSC had a peak amplitude of 39 pA. B, After 21 minutes exposure to FK960 the peak amplitude was 38 pA. C. The records recorded in control and FK960 are shown superimposed in C. The IPSC reversed at −55 mV (not shown) D, histograms showing the pooled mean IPSC data from 3 experiments before and after FK960, there was no significant difference (P=0.9).

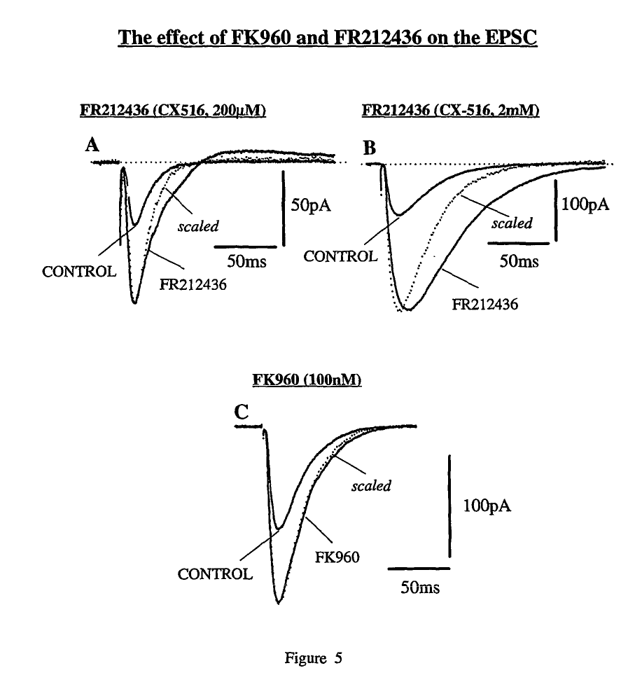

FIG. 5 shows the effect of FK960 and FR212436 on the EPSC. Excitatory post-synaptic currents (EPSCs) recorded in CA1 neurones voltage clamped at −70 mV increased in amplitude after exposure to FR212436 at 200 μM (from 42 pA to 95 pA, A) and 2 mM (from 87 pA to 247 pA, B). When the control currents were scaled (dotted line) to the same peak amplitude seen in the presence of FR212436 there was a clear slowing in the decay phase of the EPSC which was significant at the higher concentration. In contrast FK960 increased EPSC amplitude, in this neurone from 103 pA to 175 pA, without any significant change in the EPSC decay time constant (C); the scaled control trace is superimposed on the trace seen in FK960.

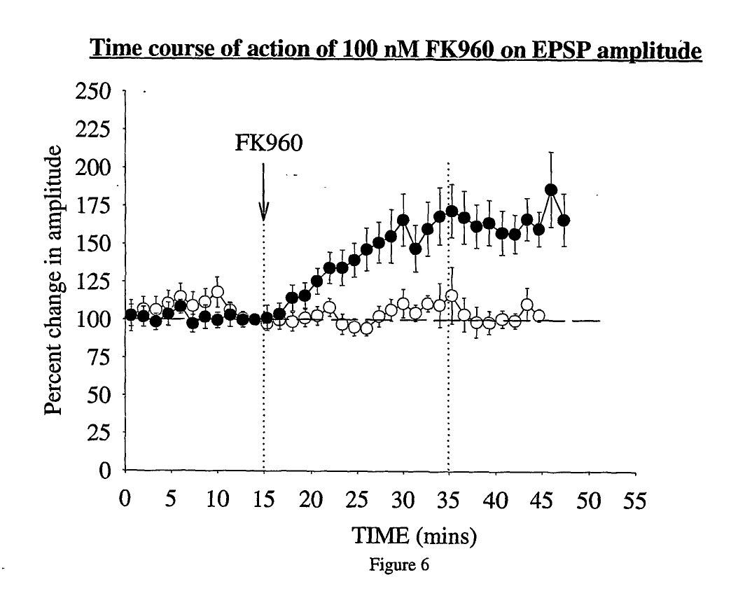

FIG. 6 shows the time course of action of FK960 on EPSP amplitude. At 15 minutes (first horizontal dotted line), the bath solution was changed from the control aCSF to a solution containing either 100 nM FK960 (filled circles, n=9 neurones) or control aCSF (open circles, n=10 neurones) and left in contact with this solution for the duration of the experiment. During the initial 15 minutes there was no difference between the two groups of neurones but on exposure to FK960 there was a progressive increase in EPSP amplitude. In contrast there was no corresponding change for the control aCSF group. The increase in amplitude seen after 21 minutes in FK960 was significantly greater than that seen in the control aCSF group.

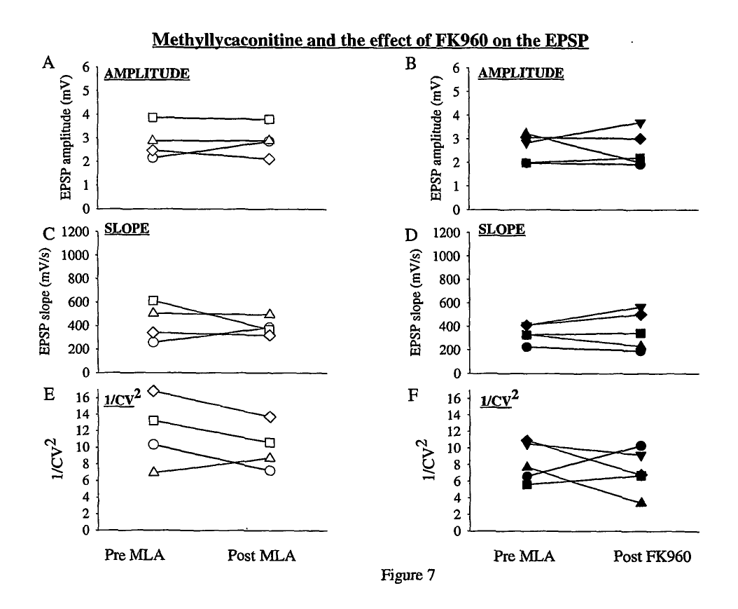

FIG. 7 shows the action of FK960 on the EPSP is blocked by pre-treatment with methyllycaconitine (MLA). EPSP amplitudes (A, B), slopes (C, D) and quantal contents (1/CV², the reciprocal of the square of the coefficient of variation, E, F) were determined before (pre-MLA) and 21 min after exposure to either FK960 (post-FK960, n=5 neurones) or MLA aCSF (post-MLA, n=4 neurones). EPSP amplitudes were measured individually and the mean of 50 consecutive EPSPs before and after changeover to either MLA aCSF (A) or FK960 (B) was determined. The slopes were determined by averaging 50 consecutive EPSPs at the appropriate times and calculating the slope of the rising phase of the averaged EPSP. The value 1/CV² (a measure of the mean number of quanta released) was determined from 100 EPSPs recorded during the 15 minutes prior to changeover and compared to the same number recorded after 21 min later in either to either MLA aCSF (E) or FK960 (F). The action of FK960 on the EPSP is abolished by MLA.

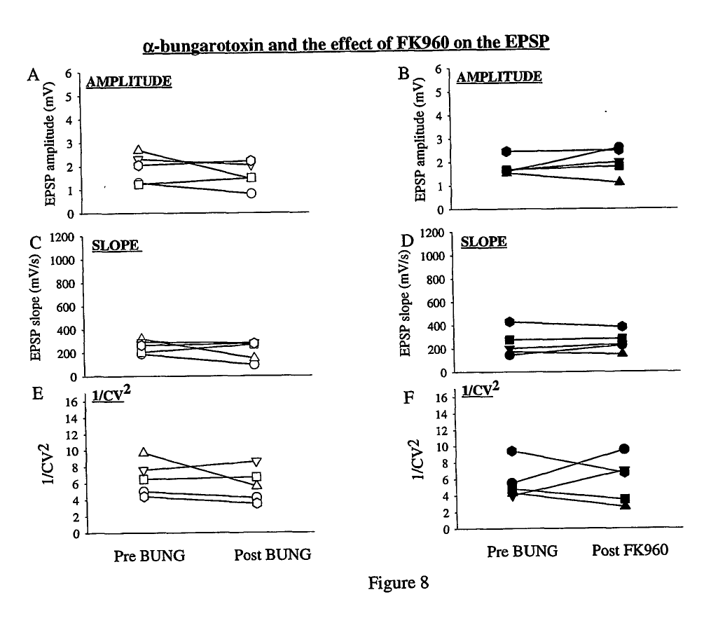

FIG. 8 shows the action of FK960 on the EPSP is attenuated by pre-treatment with α-bungarotoxin. EPSP amplitudes (A, B), slopes (C, D) and quantal contents (1/CV², the reciprocal of the square of the coefficient of variation, E, F) were determined before (pre-bung) and 21 min after exposure to either FK960 (post-FK960, n=5 neurones) or α-bungarotoxin aCSF (post-bung, n=5 neurones). EPSP amplitudes were measured individually and the mean of 50 consecutive EPSPs before and after changeover to either α-bungarotoxin aCSF (A) or FK960 (B) was determined. The slopes were determined by averaging 50 consecutive EPSPs at the appropriate times and calculating the slope of the rising phase of the averaged EPSP. The value 1/CV² (a measure of the mean number of quanta released) was determined from 100 EPSPs recorded during prior to changeover and compared to that determined from the same number of EPSPs recorded after 21 min exposure to either α-bungarotoxin aCSF (E) or FK960 (F). There was no significant action of FK960 on either EPSP amplitude or slope and no consistent action on 1/CV² in the presence of α-bungarotoxin.

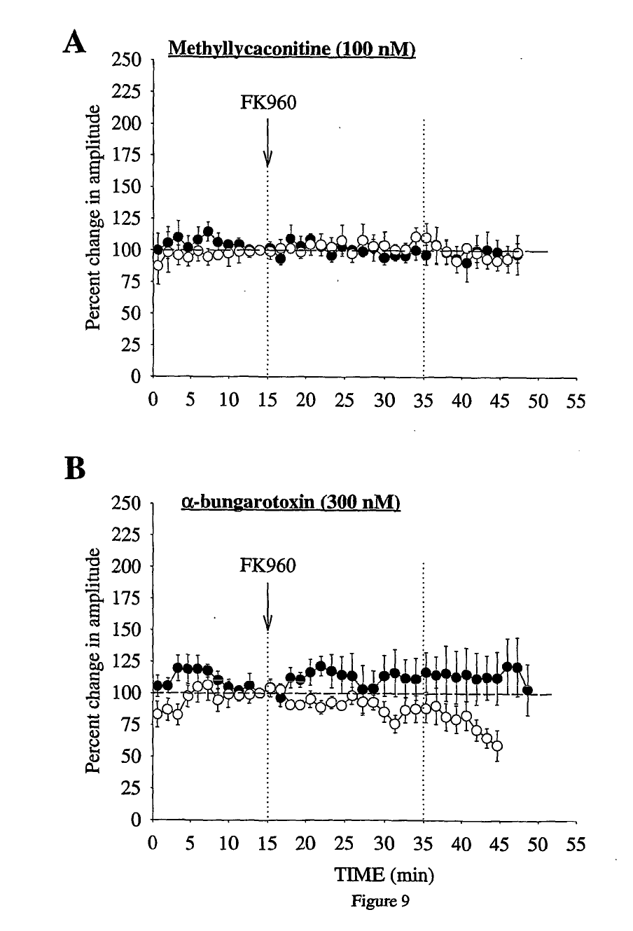

FIG. 9 shows α7nACh receptor antagonists block the action of FK960 on EPSP amplitude. A, 100 nM methyllycaconitine (MLA) aCSF was applied at t=0 minutes and remained in contact with the preparation thereafter. At t=15 minutes (indicated by first horizontal dotted line) the bathing solution was exchanged for one containing either FK960 (filled symbols, n=5 neurones) or MLA aCSF (open symbols, n=4 neurones). After 21 minutes in FK960 there was no significant difference between the 2 groups. B, similar results were obtained with 300 nM α-bungarotoxin, which also attenuated the action of FK960 on EPSP amplitude. At t=15 minutes the bathing solution was exchanged for to one containing either FK960 (filled symbols) or α-bungarotoxin aCSF (open symbols).

### EXAMPLES

### Methods

Male rats (Sprague Dawley (SD), Charles Rivers, 50-100 grams) were decapitated, the brain was removed and placed in well oxygenated artificial cerebrospinal fluid (aCSF) at 4° C. which had the following composition in mM NaCl, 126; KCl, 2.75; NaHCO₃, 26; NaH₂PO₄, 1.25; D-Glucose, 10; MgSO₄, 1.8; CaCl₂, 2.5. The brain was then placed ventral-side down on the trimming block and four cuts were made to isolate the hippocampus. The resulting block of tissue was attached using a few drops of cyanoacrylic adhesive to the platform of the tissue slicing apparatus (Vibroslice, Campden Instruments) and 450 μm thick transverse slices cut. The slices were placed in well-oxygenated aCSF at room temperature in a holding chamber and allowed to equilibrate for at least 1 hour. In experiments in which α-bungarotoxin was studied the holding chamber aCSF also contained 500 nM α-bungarotoxin. Slices were transferred to the recording chamber, also at room temperature, from 1 to 7 hours after being cut. The flow rate was 2.5-3.0 ml.min⁻¹ and was monitored throughout the experiment; data were not accepted from cells in which the flow rate fell below 2.5 ml.min⁻¹. The aCSF was gassed with 95% O₂, 5% CO₂. A single razor cut was made to isolate CA3 from the CA1 subfield. Movement of the slice was prevented by platinum weights. EPSPs were evoked using stainless-steel wire stimulating electrodes, placed on the stratum radiatum.

Patch electrodes, pulled on Narishige P83 vertical puller, were filled with the following solution; Kgluconate, 120; KCl, 10; NaCl, 5; EGTA, 10, HEPES, 10; MgCl₂, 2, CaCl₂, 1; Na₂ATP, 2; NaGTP, 1; brought to pH 7.3 with KOH. In experiments in which FK960 was compared to FR212436 (Fujisawa Pharmaceutical Co.; also known as CX516 (1-(quinoxalin-6-ylcarbonyl)piperidine, Cortex Pharmaceuticals) on the EPSC, 4 mM QX314 (Tocris, UK) was included in the pipette solution to prevent neuronal spiking. FK960 was dissolved in aCSF to give a 10 mM (2.833 mg.ml⁻¹) stock solution. Serial dilution of this stock was carried out to achieve the desired final concentration. Stock solutions of FK960 were made up fresh each day. FR212436 was dissolved in DMSO to give a 1 M stock solution, which was diluted in aCSF to achieve the final bath concentration. Methyllycaconitine (Tocris) was dissolved in 50% ethanol and α-bungarotoxin (Sigma and Calbiochem) dissolved in distilled water to give stock solutions of 100 μM which were serially diluted to achieve the final bath concentration.

Intracellular recordings were made from CA1 pyramidal neurones characterised by a sag on the voltage response to a hyperpolarising current pulse. Neurones were selected on the basis of resting potentials greater than −54 mV and action potentials greater than 70 mV. The bath solution was then changed to control aCSF, which contained picrotoxin (100 μM), bicuculline (10 μM) and D-AP5 (50 μM). The preparation was exposed to the control aCSF for 15 minutes to ensure adequate block of GABA_{A} and NMDA receptors. Input resistance of the neurone was monitored at the start and at times throughout the experiments by injecting a hyperpolarising current pulse (intensity 0.05 nA, duration 400 ms). When the action of FK960 on the EPSP was examined stimulus intensity was adjusted so that it was ‘minimal’ for evoking an EPSP. This ensured 1) only one or a small number of axons were excited and 2) the stimulus evoked EPSPs were sub-threshold for action potential discharge under control and test conditions. The frequency of nerve stimulation was 0.25 Hz. EPSPs were evoked and after 5 minutes the bath solution was changed to one of the following; control aCSF; methyllycaconitine (10 or 100 nM) in control aCSF or α-bungarotoxin (300 nM) in control aCSF for 15 minutes. In the continued presence of the pre-treatment drug the preparation was then exposed either to an aCSF to which was added either FK960 (at a concentration of 100 nM) or control aCSF containing the only the pre-treatment drug. Data were collected for at least a further 24 minutes.

Resealing of the membrane patch was indicated by a small fall in Eₘ that could be prevented either by applying slight pressure or offsetting the fall by current injection. Cells were rejected if Eₘ changed by more than 3.0 mV between t=−1 min 40 s and +21 min 40 s with reference to changeover to FK960.

EPSP amplitude was measured using either Patch and Voltage clamp or Signal software (CED, Cambridge). Two cursors were placed on each record; one cursor was positioned on the baseline before the stimulus artefact and a second on the peak of the EPSP. The slope of the rising phase of the EPSP was measured by placing cursors at 10% and 50% of peak amplitude and measuring the slope between these two points using a program written in Signal (CED, Cambridge). In a separate series of experiments CA1 neurones were voltage clamped at −70 mV in continuous mode (Axoclamp 2A, Axon Instruments) and EPSCs were evoked; series resistance was checked by applying 5 or 10 mV hyperpolarising voltage pulses at regular intervals. The time constant of the decay phase of the EPSC was determined by fitting a single exponential (Patch and voltage clamp software vers. 6.0, Cambridge Electronic Design, Cambridge UK). The GABAergic IPSC, evoked at a rate of 0.1 Hz, was also studied after block of AMPA and NMDA receptors with, respectively, 15 μM CNQX (6-Cyano-7-nitroquinoxaline-2,3-dione, Tocris, UK) and 50 μM D-AP5 (D(−)-2-Amino-5-phosphonopentanoic acid, Tocris, UK).

Quantal content (m_{cv}), a measure of pre-synaptic function, was determined by the“Variance” method (del Castillo & Katz, 1954; Martin, 1977), which is based on the idea that trial to trial variation in EPSP amplitude reflects the probabilistic organisation of the quantal release mechanism. Quantal content (m_{cv}) was determined from the 100 EPSPs recorded prior to changeover to either control aCSF or FK960 and from the same number measured after 21 minutes exposure to either FK960 or control aCSF. EPSP amplitude was measured as detailed above except that two cursors were placed on the baseline before the stimulus artefact to measure the amplitude and variance of the noise and a third placed on the peak of the EPSP, the cursors were approximately equidistant from each other. The mean (E) and standard deviation (a) of the series of EPSPs were determined. The assumption made was that release at synapses between Schaffer collateral-commissural axons and CA1 neurones, under the conditions of our experiments, conforms to Poisson's Law. Then (Martin 1977):—

m_{cv}=¹/CV² (1)

where CV=σ/E=σ²/E² (2)

after correction for the variance of the noise (σ²=σₑ²−σₙ²) where σₑ² is EPSP variance and σₙ² the variance of the noise. The calculation did not include correction for the variation in quantal size thus CV as calculated above will be somewhat greater than the CV of the quantal distribution.

No corrections were made for non-linear summation of the EPSPs, which were on average less than 6 mV.

Quantum size, a measure of the post-synaptic effect of a quantum of transmitter, was determined from the ratio of EPSP amplitude to quantal content. To determine the frequency of spontaneous EPSPs the 1 second following each stimulus was omitted from the analysis, thus only events in the 3 seconds preceding each stimulus were included in the analysis.

The experiments were carried out pseudo-randomly using a sequence generated using the RAND function in Microsoft Excel. Cells were rejected if more than 1 EPSP was supra-threshold during the sampling period. Statistical analysis of the data was performed using Sigmastat analysis software (v 2.0, Jandel). EPSP amplitude and slopes were measured before exposure to FK960 (t=−1 min 40 secs, hereafter simplified to −1 min) and after exposure (t=+21 min 40 secs, simplified to 21 min) to either FK960 in the pre-treatment solution or the pre-treatment solution to which no FK960 was added. The 21 min 40 secs time period represents the mid point of the sampling period in which 50 consecutive EPSPs were measured following a 20 min exposure to 100 nM FK960.

### Results

Intracellular recordings from CA1 neurones were usually maintained for more than 1 hour. After rupturing the cell membrane to go into whole-cell mode, and confirming the stability of the recording, the bathing solution was exchanged for one containing 50 μM D-AP5, 100 μM picrotoxin and 10 μM bicuculline (control aCSF) to block NMDA and GABA_{A} receptors. Recordings from one CA1 neurone obtained 15 min after exposure to control aCSF are shown in FIG. 1. Stimulation of the stratum radiatum resulted in EPSPs which fluctuated in amplitude; in this experiment the range was from 0.2 mV to 5.8 mV (FIG. 1C). The averaged record of 50 consecutive EPSPs in control aCSF is shown in FIG. 1A. The slope of the rising phase, determined from 10% to 50% of peak amplitude, was 326.7 mV/s. In this experiment GABA_{B} receptors were not blocked and the EPSP was followed by a late, undershooting GABA_{B} inhibitory post-synaptic potential (IPSP). The bathing solution was then exchanged for one, which contained 100 nM FK960, and over the course of the next 30 minutes there was a gradual increase in EPSP amplitude (FIG. 1B). The averaged record of 50 consecutive EPSPs recorded after 21 min in FK960 is shown superimposed on the control record in FIG. 1A. The slope of the rising phase of the EPSP increased to 742.8 mV/s and was accompanied by a corresponding increase in mean EPSP amplitude from 2.6 mV (n=50) to 5.5 mV, reflected in the rightwards shift of the amplitude histogram (FIGS. 1C, D). The quantal content (1/CV²) also increased over the course of the experiment (FIG. 1B) and rose from 6.4 to 13.4 in FK960 based on 225 EPSPs measured before exposure to FK960 and all 287 EPSPs measured after 20 mins exposure to FK960.

### (a) Dose-Response Relationship for FK960

The choice of FK960 concentration to investigate was determined in a preliminary series of experiments in which doses of 0, 50, 100 and 200 nM FK960 were examined for their effect on the slope of the excitatory post-synaptic potential (EPSP). The greatest increase in slope was seen with 100 nM FK960 (FIG. 2) which increased EPSP slope by 55% ±13 (SEM, n=5), this was significantly greater than the 11.0±10% increase seen in control aCSF (P=0.04, one-way ANOVA), none of the other groups differed significantly from control. Matsuoka & Satoh (1998) also found 100 nM FK960 significantly increased the magnitude of long-term potentiation in guinea pig hippocampus. Consequently in the experiments reported here 100 nM FK960 was used, (in some of the initial experiments 94 nM was used).

### (b) The Effect of FK960 on Membrane Potential and Input Resistance

The effect of FK960 (94 nM and 100 nM) on the passive membrane properties of CA1 neurones was examined and found to have no significant effect on either the resting membrane potential, which was −60.3 mV±0.9 (n=11) in control aCSF and −59.4 mV±0.7 in FK960 (P=0.13, paired t-test), or input resistance; 175.3 MΩ±23.8 (n=11) in control aCSF and 164.2 MΩ±221.7 in FK960 (P=0.09, paired t-test), the average exposure time was 30±2 mins.

### (c) The Effect of FK960 on the EPSP

In experiments in which hippocampal slices were exposed only to control aCSF throughout, mean EPSP amplitude was unchanged at 1.9 mV±0.2 (n=10) (FIG. 3A). In contrast, a 65% increase in mean EPSP amplitude from 2.3 mV±0.2 (n=9) to 3.8 mV±0.4 was seen in FK960 (FIG. 3B). The increase in EPSP amplitude in neurones exposed to FK960 was significantly greater than in those exposed to the control aCSF (P=0.004, one way ANOVA). In 100 nM FK960 mean EPSP slope increased by 51% from 357.8 mV/s±74.0 (n=9) to 540.5 mV/s±99.8%, (FIGS. 1, 3D) after 21 mins. In control aCSF EPSP slope decreased by 8% from 227.8 mV/s±31.4 (n=10) to 209.1 mV/s±28.2 (FIG. 3C). The increase in slope seen in FK960 was significantly greater than the increase seen in control (P=0.001, one way ANOVA).

The quantal content (1/CV²) was calculated from the mean and variance of 100 EPSP amplitudes recorded prior to exposure to either control aCSF or 100 nM FK960. 1/CV² was also determined for the same neurones from 100 EPSP amplitudes recorded after 21 minutes exposure to either control aCSF or FK960. When changeover was made to control aCSF the mean value for 1/CV² decreased by 6.5% in 10 CA1 neurones from 6.2 (range 3.2-9.0, FIG. 3E) to 5.8 (range 2.3-9.2, FIG. 3E). However, in neurones exposed to 100 nM FK960 1/CV² increased by 53% from 6.4 (range 2.8-9.6, FIG. 3F) to 9.8 (range 5.4-16.3, FIG. 3F). The changes in 1/CV² after changeover to either control aCSF or FK960 mirrored the corresponding changes in EPSP amplitude (FIGS. 1B, 3). In control aCSF mean quantum size was 0.33 mV±0.05 (n=8) and was 0.37 mV±0.05 21 mins later. In a further 7 neurones mean quantum size was 0.39 mV±0.10 and was 0.42 mV±0.10 after 21 minutes exposure to 100 nM FK960. These changes in quantal size were not significant (P>0.8, t-test).

The frequency of spontaneous EPSPs, which includes spike-dependent and spike-independent responses, in 4 neurones was 0.65 spontaneous EPSPs/sec determined for the 10 minutes before changeover to 100 nM FK960 and 0.54 spontaneous EPSP/sec for the period 20-30 min after exposure to FK960. The fall in frequency was not significant (P=0.25, paired t-test).

### (d) The Effect of FK960 on the GABAergic IPSC

To rule out the possibility that inhibition of GABA release mediates the action of FK960 on the EPSP the effect of FK960 on the inhibitory post-synaptic current (IPSC) was examined. IPSCs were elicited in neurones in voltage clamped at either −70 mV or −75 mV, after blockade of AMPA and NMDA receptors with 15 □M CNQX and 50 □M D-AP5 respectively, by stimulating the stratum radiatum. The IPSC consisted largely of a GABA_{A} component (FIG. 4); although sometimes a smaller GABA_{B} component was also present. In the experiment illustrated in FIG. 4 IPSC amplitude was 39 pA in control aCSF (FIG. 4A) and was essentially unchanged with an amplitude of 38 pA after 21 minutes in FK960 (FIGS. 4B,C). Mean IPSC amplitude in 3 experiments was 48 pA±10 in control aCSF and 46 pA±4 in FK960 (FIG. 4D), there was no significant difference (P=0.9, paired t-test).

### (e) The Effect of FK960 on the Excitatory Post-Synaptic Current (EPSC).

The increase in EPSC amplitude following exposure to FK960 was not accompanied by a significant change in the decay time constant (τ_{EPSC}) (FIG. 5C). In 4 experiments (in which 4 mM QX314 was added to the pipette solution to prevent spiking) there was an increase in EPSC amplitude from 98.3 pA±14.5 to 158.3 pA±15 (P=0.047, paired t-test) after 20-22 mins exposure to 100 nM FK960. There was an increase in τ_{EPSC} from 22.2 ms±3.1 to 28.4 ms±6.1 which was not significant (P=0.23, paired t-test).

In experiments in which the slice was exposed to 2 mM FR212436 for 5-10 min EPSC amplitude increased from 73.7 pA±8.9 (n=3) to 239.7 pA±49.7 and τ_{EPSC} increased significantly (P=0.046, paired t-test) from 21.5 ms±3.7 to 46.7 ms±5.4 (FIG. 5B). When exposed to 200 μM FR212436 for 20 min EPSC amplitude increased from 84 pA±36.6 (n=3) to 181.3 pA±46.8 and τ_{EPSC} increased from 15.0 ms±2.2 to 22.4 ms±5.0 although neither change quite reached statistical significance (P>0.2, paired t-test) (FIG. 5A).

### (f) Time Course of Changes in EPSP Amplitude

EPSP amplitude increased with time after exposure to FK960 and this increase was maintained for times in excess of 21 minutes. (FIGS. 1, 6). Intracellular recording could not be maintained for the same length of time in all neurones and so complete data for all neurones were not available for longer time periods.

There was a 64% increase in EPSP amplitude, from 2.2 mV±0.3 (n=7) to 3.6 mV±0.5 after 27 mins in FK960. At this time mean EPSP amplitude fell by 5% in the control aCSF group from 2.0 mV±0.3 (SEM, n=7) to 1.9 mV±0.3. The change in EPSP amplitude was significantly greater for the FK960 group over the control aCSF group (P=0.03, t-test).

### g) Effect of FK960 on EPSP Amplitude in the Presence of α7 Nicotinic ACh Receptor Antagonists

The α7nACh receptor antagonists methyllycaconitine (at concentrations of 10 and 100 nMand α-bungarotoxin (at a concentration of 300 nM) were studied for their action on the enhancement of the EPSP by FK960. α-bungarotoxin (100 nM) has been shown to block the action of nicotine on central neurones (McGehee et al, 1995; Gray et al, 1996). In the experiments reported here 300 nM α-bungarotoxin was used to ensure rapid block during the 15 minute pre-treatment period.

The α7nACh receptor antagonist methyllycaconitine (MLA) at a concentration of 100 nM not only completely blocked the action of FK960 on the EPSP but appeared to be without any action on its own (FIGS. 7, 9A). In 4 CA1 neurones mean EPSP amplitude remained unchanged at 2.9 mV±0.4 after 21 minutes in MLA aCSF (FIGS. 7A, 9A). In another 5 CA1 neurones mean EPSP amplitude was unchanged at 2.6 mV±0.3 after 21 minutes exposure to 100 nM FK960 in MLA (FIGS. 7B, 9A). Clearly none of these values were significantly different. Overall EPSP slope and 1/CV² values were similarly unchanged (FIG. 7).

When 10 nM MLA was employed EPSP amplitude fell by 5% from 2.2 mV±0.2 (n=3) after 21 minutes in MLA aCSF but rose by 10% from a mean of 3.1 mV±0.4 (n=3) when FK960 was included.

Mean EPSP amplitude fell from 1.9 mV±0.3 (n=5) to 1.6 mV±0.3 after 21 minutes in α-bungarotoxin aCSF (FIGS. 8A, 9B). In another 5 CA1 neurones mean EPSP amplitude increased by 11% from 1.8 mV±0.2 in α-bungarotoxin aCSF to 2.0 mV±0.3 in FK960 (FIGS. 8B, 9B). These changes in mean EPSP amplitude in the two groups of experiments were not significantly different (P>0.05, one way ANOVA). The changes in amplitude seen in individual neurones exposed to either α-bungarotoxin aCSF or FK960 were reflected in the corresponding changes in EPSP slope and 1/CV² (FIG. 8). The monotonous increase in amplitude seen when FK960 was added to the bath (FIGS. 1, 6) was clearly attenuated when the hippocampal slices were pre-treated with MLA (FIG. 9A). The attenuation was slightly less marked when the slices were pre-treated with α-bungarotoxin (FIG. 9B), however FK960 had no significant action on EPSP amplitude in the presence of α-bungarotoxin (P>0.05, one-way ANOVA). An additional feature of the action of α-bungarotoxin was that on its own it appeared to cause a reduction in EPSP amplitude at times>40 minutes after introduction into the bath (FIG. 9B).

Statistical analysis of the pooled EPSP amplitude data indicated that, in the control aCSF pre-treatment group, there was a significant difference between data obtained from neurones exposed for 21 min to FK960 and those exposed to control ACSF for the same time period (P=0.001, one-way ANOVA).

The present inventors have thus demonstrated that the increase in EPSP amplitude and slope seen in hippocampal CA1 neurones following exposure to FK960 can be accounted for by an increase in transmitter release. FK960 had no significant effect either on the passive membrane properties of CA1 neurones or on the decay phase of the EPSC. The action of FK960 on the decay time constant (τ_{EPSC}) of the EPSC was examined and compared to that of the cognitive enhancer FR212436 (CX516) (Arai et al 1996a,b; Arai & Lynch, 1998). They confirmed that FR212436 at millimolar concentrations increased τ_{EPSC} but were unable to demonstrate a similar effect for 100 nM FK960 on τ_{EPSC}. Receptor properties such as affinity and the kinetics of desensitisation and deactivation are important factors controlling the amplitude and time course of synaptic current at fast excitatory AMPAergic synapses (Edmonds et al, 1995; Jones and Westbrook, 1996); The AMPAkine benzoylpyrrolidine compounds, CX516 and BDP-20, (Arai, et al, 1996a,b; Arai & Lynch, 1998) and aniracetam (Tang et al, 1991; Isaacson & Nicoll, 1991) increase the amplitude and duration of the excitatory post-synaptic current (EPSC) and field potential (fEPSP) by reducing receptor desensitisation and/or slowing de-activation (Arai & Lynch, 1998). Interestingly thiocyanate increases AMPA binding affinity and promotes desensitisation by enhancing conversion to the desensitised state, thereby reducing AMPA receptor currents in excised patches (Arai et al 1995). However, it seems that compounds such as cyclothiazide that dramatically reduce desensitisation (Vyklicky, et al, 1991; Yamada & Tang, 1993) may, in some situations, have little effect on synaptic currents by this mechanism alone (Arai & Lynch, 1998). Cyclothiazide also acts pre-synaptically to increase glutamate release (Vyklicky, et al, 1991; Bellingham & Walmsley, 1999). The finding in this study that FK960 had no effect on τ_{EPSC} and the observation that quantal size, determined indirectly, did not change significantly suggests that FK960 does not alter the properties of post-synaptic AMPAergic glutamate receptors on CA1 neurones. These findings rule out a significant post-synaptic action for FK960.

There existed the possibility that FK960 may exert its action by inhibiting GABA release, thus relieving the glutamatergic nerve terminals from a tonic inhibition. It seems that this is an unlikely mechanism since FK960 did not significantly alter the amplitude (nor apparently the decay time constant (τ_{IPSC}) see FIG. 4) of the IPSC.

Quantal analysis of the EPSP, employing the variance method (del Castillo & Katz, 1954; Martin, 1977), supports the idea that FK960 acts on the nerve terminal to increase transmitter release. FK960 caused a greater increase in the quantal content of the EPSP than did exposure to control aCSF. Neither treatment was accompanied by any significant change in quantum size. There were variations in the degree to which quantal content increased following exposure to FK960 which may be a result of the time required for FK960 to reach an effective concentration at its site of action or it may be a consequence of the characteristic dose/response relationship for FK960. In experiments in which extracellular recording of population spikes was employed, which allow much longer periods of recording than usually permitted with the ‘blind patch’ technique, it appears that FK960 continues to enhance the size of the population spike for up to 2 hours (Matsuyama et al, 2000).

There was no significant change in the frequency of spontaneous EPSPs at times (21 mins exposure) when FK960 significantly increased evoked EPSP amplitude suggesting that FK960 has no action on baseline transmitter release from nerve terminals. To confirm this it will be necessary to determine miniature EPSP frequency in the presence of the sodium channel blocker tetrodotoxin to block all spike-dependent transmitter release.

A further observation is that the action of FK960 was blocked by methyllycaconitine (Macallan et al, 1988), and α-bungarotoxin indicating that activation of the α7 subtype of nicotinic acetylcholine (α7nACh) receptor is an obligatory link in the action of FK960. The α7nACh receptor (Couturier et al, 1990; McGehee et al, 1995; McGehee & Role 1995; Gray et al, 1996) is known to be widely distributed in the mammalian CNS (Seguela et al, 1993; MacDermott, 1999; Whiteaker, et al, 1999) probably in homomeric form (Seguela et al, 1993; McGehee & Role 1995; MacDermott et al, 1999; but see Yum et al, 1996; Albuquerque et al, 1997b; Wonnacott, 1997).

The α7nACh receptor is of particular interest because it is highly permeable to calcium ions (McGhee & Role, 1995; McGhee at al, 1995; Gray et al, 1996), has a P_{Ca}/P_{Na} in the order of 20 (Seguela et al, 1993), is located pre-synaptically (McGehee et al, 1995; Alkondon et al, 1996; Gray et al, 1996) in the hippocampus as well as a number of other brain areas (McGhee & Role, 1995; MacDermott et al, 1999) and is, therefore, likely to play an important role in modulation of transmitter release by ACh (McGehee et al, 1995; Alkondon et al, 1996; Gray et al, 1996;Wonnacott, 1997, Radcliffe & Dani, 1998; MacDermott et al, 1999). However, at present the source of ACh involved in such an action, whether from septo-hippocampal afferents or local interneurones, is unclear (Alkondon et al, 1998; Vizi, 2000). It should be noted that the septal cholinergic innervation of the hippocampus should be complete or nearly complete in the rats used in this study which were 50-100 gm in weight (21-34 days after birth, Milner et al, 1983).

Recently post-synaptic α7nACh receptors have been described (Frazier et al, 1998a, b; Alkondon et al, 1998) which may behave differently from pre-synaptic α7nACh receptors during recovery from desensitisation (McGehee et al, 1995; Khiroug et al, 1998; Frazier et al, 1998b). However, in a number of assays FK960 failed to displace radiolabelled ligands with high affinity to cholinergic receptors and showed no other cholinergic activity.

It is well known that neurotransmitter gated ion channels opened by glutamate and GABA can be further regulated by other modulators; for example, steroids and barbiturates in the case of GABA_{A} receptors and glycine and polyamines in the case of NMDA receptors. It seems that α7nACh receptors are also amenable to modulation via a site on the α7 subunit (Schrattenholz et al, 1996; Albuquerque et al, 1997a). The present experiments do not enable a conclusion to be drawn as to whether or not FK960 acts at this site but they do not rule out the possibility that FK960 may exert its action by modulating the strength of nicotinic transmission via such a mechanism. One approach to test this would be to see if the enhancement of EPSC amplitude and mEPSC frequency by nicotine in CA1 neurones is further increased by FK960 and whether this is blocked by the monoclonal antibody FK1 (Schrattenholz et al, 1996). However, the action of FK960 on the EPSP develops only slowly, over the course of tens of minutes, before a maximum effect is reached which may not be compatible with such a direct action.

The present inventors also found, in agreement with others, that the dose-response relationship for FK960 is bell-shaped (Yamazaki et al, 1996; Matsuoka & Satoh, 1998; Matsuyama, et al 2000). It is not possible to determine whether higher concentrations of FK960 act at a site distinct from that targeted by lower concentrations, the experiments reported here do not address this question. It is interesting to note that the bell-shaped dose-response relationship for FK960 is seen in experiments on isolated brain slices (Matsuoka & Satoh, 1998; Matsuyama, et al, 2000) and intact animals (Yamazaki et al, 1996; Matsuyama, et al, 2000). It is also worth noting vis a vis a putative role for FK960 at α7nACh receptors that galanthamine (and 5-HT) which acts at a site on this receptor also has a bell-shaped dose-response relationship (normalised ACh current amplitude vs 1-methyl-galanthamine concentration, Schrattenholz et al, 1996). In contrast, the dose-response relationship for the cognitive enhancer CX516 (FR21243), (% increase in peak current vs CX-516 concentration) has a sigmoidal shape (Arai et al, 1996b).

One hypothesis put forward for the mechanism of action of FK960 is that, directly or indirectly, it activates somatostatinergic neurones (Yamazaki et al, 1996; Matsuoka & Satoh, 1998). These experiments showed that pre-treatment of animals with cysteamine, which reduces somatostatin levels in the brain (Yamazaki et al, 1996) significantly attenuated the action of FK960 both in vitro (LTP in brain slices, Matsuoka & Satoh, 1998) and in vivo (reversal of scopolamine-induced impairment using passive avoidance and Morris water maze techniques, Yamazaki et al, 1996). Lesion experiments were included in the study of Yamazaki et al, 1996 which led to the further suggestion that serotinergic and, perhaps not surprisingly, cholinergic systems were involved in the action of FK960 although the detailed nature of the relationship between these transmitter systems still remains to be worked out. The present study, in addition, implicates glutamatergic synapses in the growing collection of transmitter systems at which FK960 appears to be active.

The long slow time course with which the action of FK960 on transmitter release develops is of considerable interest. Clearly the mechanism could be related to the kinetics with which FK960 enters the nerve terminal in order to interact with an intracellular receptor. However, this is unlikely given the ease with which FK960 is orally absorbed and enters the brain. An alternative hypothesis would be that FK960 interacts with a specific receptor to cause a build-up of an intracellular messenger or interfere with a constitutively active enzyme cascade and cause a gradual change in the levels of a phosphorylated product that regulates transmitter release. Recently a similar slow increase in quantal transmitter release at a glutamatergic synapse, following adrenergic stimulation, has been reported. The mechanism involves activation of a nitric oxide-independent guanylyl cyclase, resulting in an accumulation of intraterminal cyclic GMP and activation of protein kinase G (Yawo, 1999).

In conclusion, the data presented here show that FK960 increased the number of quanta released in response to nerve impulses thereby increasing the amplitude and slope of the EPSP with no effect on quantum size. The importance of this positive modulation of AMPAergic transmission in relation to other transmitter systems with regard to enhanced cognitive performance is unknown, although we have demonstrated that activation of α7nACh receptors is required for this particular action of FK960. It is well established that up-regulation of AMPAergic transmission by, for example, AMPAkines is sufficient in some circumstances to promote significant improvement in cognitive function in man (Lynch et al, 1996). Therefore, it should not to be unexpected that enhanced glutamatergic transmission by FK960 will contribute, either directly or indirectly, a therapeutic benefit in memory performance.

### References

ALBUQUEQUE, E. X., ALKONDON, M., PEREIRA, E. F., CASTRO, N. G., SCHRATTENHOLZ, A., BARBOSA, C. T. F., BONFANTE-CABARCAS, R., ARACAVA, Y., EISENBERG, H. M. & MAELICKE, A. (1997a). Properties of neuronal nicotinic acetylcholine receptors: Pharmacological characterization and modulation of synaptic function. J.Pharmac.Exp.Therap. 280, 1117-1136.

ALBUQUEQUE, E. X., PEREIRA, E. F., ALKONDON, M., SCHRATTENHOLZ, A. & MAELICKE, A. (1997b). Nicotinic acetylcholine receptors on hippocampal neurons: distribution on the neuronal surface and modulation of receptor activity. J.Recept.Signal Transduct.Res. 17, 243-266.

ALKONDON, M., ROCHA, E. S., MAELICKE, A. & ALBUQUEQUE, E. X. (1996). Diversity of nicotinic acetylcholine receptors in rat brain. V. alpha-Bungarotoxin-sensitive nicotinic receptors in olfactory bulb neurones and presynaptic modulation of glutamate release. J.Pharmac.Exp.Therap. 278, 1460-1471.

ALKONDON, M., PEREIRA, E. F. & ALBUQUEQUE, E. X. (1998). α-Bungarotoxin- and methyllycaconitine-sensitive nicotinic receptors mediate fast synaptic transmission in interneurones of rat hippocampal slices. Brain Res. 810, 257-263.

ARAI, A., SILBERG, J., KESSLER, M. & LYNCH, G. (1995). Effect of thiocyanate on AMPA receptor mediated responses in excised patches and hippocampal slices. Neuroscience 66, 815-827.

ARAI, A., KESSLER, M., AMBROS-INGERSON, J., QUAN, A., YIGITER, E., ROGERS, G. & LYNCH, G. (1996a). Effects of a centrally active benzoylpyrrolidine drug on AMPA receptor kinetics. Neuroscience 75, 573-585.

ARAI, A., KESSLER, M., ROGERS, G. & LYNCH, G. (1996b). Effects of a memory-enhancing drug on DL-α-amino-3-hydroxy-5-methyl-4-isoxazolepropionic acid receptor currents and synaptic transmission in hippocampus. J.Pharmac.Exp.Therap. 278, 627-638.

ARAI, A. & LYNCH, G. (1996). Response to repetitive stimulation of AMPA receptors in patches excised from fields CA1 and CA3 of the hippocampus. Brain Res. 716, 202-206.

ARAI, A. & LYNCH, G. (1998). The waveform of synaptic transmission at hippocampal synapses is not determined by AMPA receptor desensitization. Brain Res. 799, 230-234.

ARAUJO, D. M., LAPCHAK, P. A., ROBITAILLE, Y., GAUTHIER, S. & QUIRION, R. (1988). Differential alteration of various cholinergic markers in cortical and subcortical regions of human brain in Alzheimer's Disease. J.Neurochem 50, 1914-1923.

BARTUS, R. T., DEAN, R. L., BEER, B. & LIPPA, A. S. (1982). The cholinergic hypothesis of geriatric memory dysfunction. Science 217, 408-417.

BELLINGHAM, M. C. & WALMSLEY, B. (1999). A novel presynaptic inhibitory mechanism underlies paired pulse depression at a fast central synapse. Neuron 23, 159-170.

CHORVAT, R. J., EARL, R. A. & ZACZEK, R. (1995). Acetylcholine release enhancing agents: potential therapeutics for Alzheimer's disease. Drugs of the Future 20, 1145-1162.

COUTURIER, S., BERTRAND, D., MATTER, J. M., HERNANDEZ, M. C., BERTRAND, S., MILLAR, N., VALERA, S., BARKAS, T. & BALLIVET, M. (1990). A neuronal nicotinic acetylcholine receptor subunit (α7) is developmentally regulated and forms a homo-oligomeric channel blocked by α-BTX. Neuron 5, 847-856.

DAVIES, P. & MALONEY, A. J. R. (1976). Selective loss of central cholinergic neurones in Alzheimer's Disease. Lancet2, 1403-1405.

DEL CASTILLO, J. & KATZ, B. (1954). Quantal components of the end-plate potential. J.Physiol. 124, 560-573.

EDMONDS, B., GIBB, A. J. & COLQUHOUN, D. (1995). Mechanisms of activation of glutamate receptors and the time course of excitatory syanptic currents. Annu.Rev.Physiol. 57, 495-519.

FIBIGER, H. C. (1991). Cholinergic mechanisms in learning, memory and dementia: a review of recent evidence. Trends Neurosci. 14, 220-223.

FRANCIS, P. T., PALMER, A. M., SNAPE, M. & WILCOCK, G. K. (1999). The cholinergic hypothesis of Alzheimer's Disease; a review of progress. J.Neurol.Neurosurg.Psychiatry 66, 137-147.

FRAZIER, C. J., BUHLER, A. V., WEINER, J. L. & DUNWIDDIE, T. V. (1998a). Synaptic potentials mediated via α-bungarotoxin-sensitive nicotinic acetylcholine receptors in rat hippocampal interneurones. J.Neurosci. 18, 8228-8235.

FRAZIER, C. J., ROLLINS, Y. D., BREESE, C. R., LEONARD, S., FREEDMAN, R. & DUNWIDDIE, T. V. (1998b). Acetylcholine activates an α-bungarotoxin-sensitive nicotinic current in rat hippocampal interneurones, but not pyramidal cells. J.Neurosci. 18, 1187-1195.

GALLAGHER, M. & COLOMBO, P. J. (1995). Ageing: the cholinergic hypothesis of cognitive decline. Curr.Opin.Biol. 5, 161-168.

GRANGER, R., DEADWYLER, S., DAVIS, M., MOSKOVITZ, B., KESSLER, M., ROGERS, G. & LYNCH, G. (1996). Facilitation of glutamate receptors reverses an age-associated memory impairment in rats. Synapse 22, 332-337.

GRAY, R., RAJAN, A. S., RADCLIFFE, K. A., YAKEHIRO, M. & DANI, J. A. (1996). Hippocampal synaptic transmission enhanced by low concentrations of nicotine. Nature 383, 713-716.

ISAACSON, J. & NICOLL, R. A. (1991). Aniracetam reduces glutamate receptor desensitization and slows the decay of fast excitatory synaptic currents in the hippocampus. Proc.Natl.Acad.Sci. 88, 10936-10940.

JONES, M. V. & WESTBROOK, G. L. (1996). The impact of receptor desensitization on fast synaptic transmission. Trends Neurosci. 19, 96-101.

KASA, P., RAKONCZAY, Z. & GULYA, K. (2000). The Cholinergic System in Alzheimer's Disease. Prog. Neurobiol. 52, 511-535.

KELLY, J. S. (1999). Alzheimer's disease:the tacrine legacy. Trends Pharmacol. Sci. 20, 127-129.

KHIROUG, L., SOKOLOVA, E., GINIATULLIN, R., AFZALOV, R. & NISTRI, A. (1998). Recovery from desensitization of neuronal nicotinic acetylcholine receptors of rat chromaffin cells is modulated by intracellular calcium through distinct second messengers. J.Neurosci. 18, 2458-2466.

LYNCH, G., KESSLER, M., ROGERS, G., AMBROS-INGERSON, J. & GRANGER, R. (1996). Psychological effects of a drug that facilitates brain AMPA receptors. Int.Clin.Psychopharm. 11, 13-19.

MACALLAN, D. R., LUNT, G. G., WONNACOTT, S. S., SWANSON, K. L., RAPOPORT, H. & ALBUQUERQUE, E. X. (1988). Methyllycaconitine and (+)anatoxin-a differentiate between nicotinic receptors in vertebrate and invertebrate nervous systems. FEBS Lett, 226, 357-363.

MACDERMOTT, A. B., ROLE, L. & SIEGELBAUM, S. (1999). Presynaptic ionotropic receptors and the control of transmitter release. Annu.Rev.Neurosci. 22, 443-485.

MARTIN, A. R. (1977). Junctional Transmission II. Presynaptic mechanisms. In Handbook of Physiology, eds. BROOKHART, J. M. & MOUNTCASTLE, V. B. pp. 329-355. Bethesda, Md.: American Physiological Soc.

MATSUOKA, M. & SATOH, M. (1998). FK960, a novel potential anti-dementia drug, augments long-term potentiation in mossy fiber-CA3 pathway of guinea-pig hippocampal slices. Brain Res. 794, 248-254.

MATSUYAMA, S., NOMURA, T. & NISHIZAKI, T. (2000). Sustained facilitatory action of FK960 on hippocampal neurotransmission. Brain Res. 857, 317-320.

MCGEHEE, D. S., HEATH, M. J. S., GELBER, S., DEVAY, P. & ROLE, L. W. (1995). Nicotine enhancemant of fast synaptic transmission in CNS by presynaptic receptors. Science 269, 1692-1696.

MCGEHEE, D. S. & ROLE, L. W. (1995). Physiological diversity of nicotinic acetylcholine receptors expressed by vertebrate neurons. Annu.Rev.Physiol. 57, 521-546.

MILNER, T. A., LOT, R. & AMARAL, D. G. (1983). An anatomical study of the development of the septo-hippocampal projection in the rat. Brain Res.Dev Brain Research 8, 343-371.

MUCKE, H. A. M. (1998). Drug treatments for Alzheimer's disease. Pharmaceut.News 5, 10-14.

PALMER, A. M. (1996). Neurochemical studies of Alzheimer's Disease. Neurodegeneration 5, 381-391.

RADCLIFFE, K. A. & DANI, J. A. (1998). Nicotinic stimulation produces multiple forms of increased glutamatergic synaptic transmission. J.Neurosci. 18, 7075-7083.

SCHRATTENHOLZ, A., PEREIRA, E. F., ROTH, U., WEBER, K., ALBUQUEQUE, E. X. & MAELICKE, A. (1996). Agonist responses of neuronal nicotinic acetylcholine receptors are potentiated by a novel class of allosterically acting ligands. Mol Pharmacol. 49, 1-6.

SEGUELA, P., WADICHE, J., DINELY-MILLER, K., DANI, J. A. & PATRICK, J. W. (1993). Molecular cloning, functional properties and distribution of rat brain α7: A nicotinic cation channel highly permeable to calcium. J.Neurosci. 13, 596-604.

SIRVIO, J., LARSON, J., QUACH, C. N., ROGERS, G. A. & LYNCH, G. (1996). Effects of pharmacologically facilitating glutamatergic transmission in the trisynaptic intrahippocampal circuit. Neuroscience 74, 1025-1035.

STAUBLI, U., PEREZ, Y., FANGBO, X., ROGERS, G., INGVAR, M., STONE-ELANDER, S. & LYNCH, G. (1994). Centrally active modulators of glutamate receptors facilitate the induction of long-term potentiation in vivo. Proc.Natl.Acad.Sci. 91, 11158-11162.

TANG, C. M., SHI, Q. Y., KATCHMAN, A. & LYNCH, G. (1991). Modulation of the time course of fast EPSCs and glutamate channel kinetics by aniracetam. Science 254, 288-290.

VIZI, E. S. (2000). Role of high affinity receptors and membrane transporters in nonspecific communication and drug action in the central nervous system. Pharmacol. Rev. 52, 63-89.

VYKLICKY, L., PATNEAU, D. K. & MAYER, M. L. (1991). Modulation of excitatory synaptic transmission by drugs that reduce desensitization at AMPA/kainate receptors. Neuron 7, 971-984.

WHITEAKER, P., DAVIES, A. R. L., MARKS, M. J., BLAGBROUGH, I. S., POTTER, B. V. L., WOLSTENHOLME, A. J., COLLINS, A. C. & WONNACOTT, S. (1999). An autoradiographic study of the distribution of binding sites for the novel α7-selective nicotinic radioligand [³H]-methyllycaconitine in the mouse brain. European Journal of Neuroscience 11, 2689-2696.

WONNACOTT, S. (1997). Presynaptic nicotinic ACh receptors. Trends Neurosci. 20, 92-98.

YAMADA, K. A. & TANG, C. M. (1993). Benzothiadiazides inhibit rapid glutamate receptor desensitization and enhance glutamatergic synaptic currents. J.Neurosci. 13, 3904-3915.

YAMAZAKI, M., MATSUOKA, M., MAEDA, N., OKHUBO, Y. & YAMAGUCHI, I. (1996). FK960 N-(4-Acetyl-1-piperazinyl)-p-fluorobenzamide monohydrate ameliorates the memory deficits in rats through a novel mechanism of action. J.Pharmac.Exp.Therap. 279, 1157-1173.

YAWO, H. (1999). Involvement of cGMP-dependent protein kinase in adrenergic potentiation of transmitter release from calyx-type presynaptic terminal. J.Neurosci 19, 5293-5300.

YUM, L., WOLF, K. M. & CHIAPPINELLI, V. A. (1996). Nicotinic acetylcholine receptors in separate brain regions exhibit different affinities for methyllycaconitine. Neuroscience 72, 545-555.

1. An agent for modulating excitatory synaptic transmission, which comprises a compound having α7 nicotinic acetylcholine receptor activation property as an active ingredient.

2. The agent for modulating excitatory synaptic transmission of claim 1, wherein the compound has the following formula [I]: 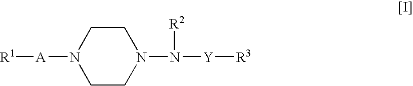
wherein
R¹ is lower alkyl, aryl, ar(lower)alkoxy or heterocyclic group, each of which may be substituted with halogen,
R² is hydrogen atom or lower alkyl,
R³ is cyclo(lower)alkyl, aryl or ar(lower)alkyl, each of which may be substituted with halogen,
A is —CO—, —SO₂— or lower alkylene, and
Y is —CO—, —SO₂— or —CONH— or pharmaceutically acceptable salts thereof

3. The agent for modulating excitatory synaptic transmission of claim 1, wherein the compound has the following formula [II-1]: 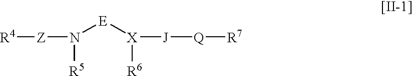
wherein
R⁴ is acyl,
R⁷ is lower alkyl, lower alkoxy, lower alkylamino, lower alkenyl, lower alkenyloxy, lower alkenylamino, lower alkynyl, lower alkynyloxy, lower alkynylamino, cyclo(lower)alkyl, cyclo(lower)alkyloxy, cyclo(lower)alkylamino, aryl, aryloxy, arylamino, a heterocyclic group or amino substituted with a heterocyclic group, each of which may be substituted with suitable substituent(s); or acyl;
Z is a single bond, —CO— or —SO₂—,
E is lower alkylene optionally substituted with suitable substituent(s),
X is CH or N,
J is a single bond, lower alkylene or 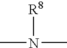
wherein R⁸ is hydrogen, lower alkyl, substituted-lower alkyl, an N-protective group, aryl, acyl or a heterocyclic group,
Q is —CH₂—, —CO—, —SO₂— or —N═CH—, and
R⁵ and R⁶ are each hydrogen or lower alkyl, or are taken together to form lower alkylene optionally condensed with a cyclic hydrocarbon or a heterocyclic ring, provided that when X is N, then
1) J is a single bond, and Q is —CH₂—, —CO— or —SO₂—, or
2) j is lower alkylene,
or pharmaceutically acceptable salts thereof.

4. The agent for modulating excitatory synaptic transmission of claim 1, wherein the compound has the following formula [II-2]: 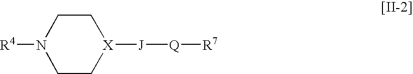
wherein
R⁴ is acyl,
R⁷ is aryl, aryloxy or arylamino, the aryl moiety of all of which may be substituted with halogen; pyridyl; or pyridylamino;
X is CH or N,
J is a single bond, lower alkylene or 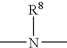
wherein R⁸ is hydrogen, lower alkyl or an N-protective group,
Q is —CH₂—, —CO— or —SO₂—, provided that when X is N, then J is a single bond or lower alkylene, or pharmaceutically acceptable salts thereof.

5. The agent for modulating excitatory synaptic transmission of any of claim 1 to claim 4, which is an agent for the prophylaxis or treatment of cerebral diseases.

6. The agent for modulating excitatory synaptic transmission of claim 5, which is an agent for the prophylaxis or treatment of dementia or amnesia.

7. A method for modulating excitatory synaptic transmission, comprising administering an effective amount of a compound having α7 nicotinic acetylcholine receptor activation property.

8. The method for modulating excitatory synaptic transmission of claim 7, wherein the compound has the following formula [I]: 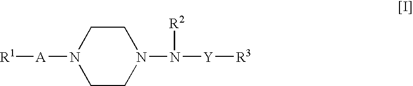
wherein
R¹ is lower alkyl, aryl, ar(lower)alkoxy or heterocyclic group, each of which may be substituted with halogen,
R² is hydrogen atom or lower alkyl,
R³ is cyclo(lower)alkyl, aryl or ar(lower)alkyl, each of which may be substituted with halogen,
A is —CO—, —SO₂— or lower alkylene, and
Y is —CO—, —SO₂— or —CONH— or pharmaceutically acceptable salts thereof

9. The method for modulating excitatory synaptic transmission of claim 7, wherein the compound has the following formula [II-1]: 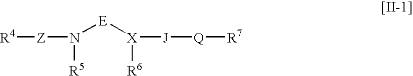
wherein
R⁴ is acyl,
R⁷ is lower alkyl, lower alkoxy, lower alkylamino, lower alkenyl, lower alkenyloxy, lower alkenylamino, lower alkynyl, lower alkynyloxy, lower alkynylamino, cyclo(lower)alkyl, cyclo(lower)alkyloxy, cyclo(lower)alkylamino, aryl, aryloxy, arylamino, a heterocyclic group or amino substituted with a heterocyclic group, each of which may be substituted with suitable substituent(s); or acyl;
Z is a single bond, —CO— or —SO₂—,
E is lower alkylene optionally substituted with suitable substituent(s),
X is CH or N,
J is a single bond, lower alkylene or 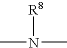
wherein R⁸ is hydrogen, lower alkyl, substituted-lower alkyl, an N-protective group, aryl, acyl or a heterocyclic group,
Q is —CH₂—, —CO—, —SO₂— or —N═CH—, and
R⁵ and R⁶ are each hydrogen or lower alkyl, or are taken together to form lower alkylene optionally condensed with a cyclic hydrocarbon or a heterocyclic ring, provided that when X is N, then
1) J is a single bond, and Q is —CH₂—, —CO— or —SO₂—, or
2) J is lower alkylene,
or pharmaceutically acceptable salts thereof.

10. The method for modulating excitatory synaptic transmission of claim 7, wherein the compound has the following formula [II-2]: 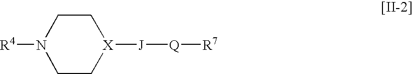
wherein
R⁴ is acyl,
R⁷ is aryl, aryloxy or arylamino, the aryl moiety of all of which may be substituted with halogen; pyridyl; or pyridylamino;
X is CH or N,
J is a single bond, lower alkylene or 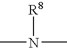
wherein R⁸ is hydrogen, lower alkyl or an N-protective group,
Q is —CH₂—, —CO— or —SO₂—, provided that when X is N, then J is a single bond or lower alkylene, or pharmaceutically acceptable salts thereof.

11. The method for modulating excitatory synaptic transmission of any of claim 7 to claim 10, which is a method for the prophylaxis or treatment of cerebral diseases.

12. The method for modulating excitatory synaptic transmission of claim 11, which is a method for the prophylaxis and/or treatment of dementia or amnesia.

13. Use of a compound having α7 nicotinic acetylcholine receptor activation property for the production of an agent for modulating excitatory synaptic transmission.

14. The use of a compound having α7 nicotinic acetylcholine receptor activation property according to claim 13, wherein the compound has the following formula [I]: 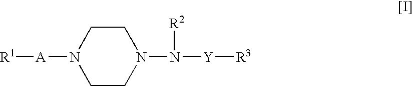
wherein
R¹ is lower alkyl, aryl, ar(lower)alkoxy or heterocyclic group, each of which may be substituted with halogen,
R² is hydrogen atom or lower alkyl,
R³ is cyclo(lower)alkyl, aryl or ar(lower)alkyl, each of which may be substituted with halogen,
A is —CO—, —SO₂— or lower alkylene, and
Y is —CO—, —SO₂— or —CONH— or pharmaceutically acceptable salts thereof.

15. The use of a compound having α7 nicotinic acetylcholine receptor activation property according to claim 13, wherein the compound has the following formula [II-1]: 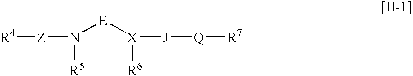
wherein
R⁴ is acyl,
R⁷ is lower alkyl, lower alkoxy, lower alkylamino, lower alkenyl, lower alkenyloxy, lower alkenylamino, lower alkynyl, lower alkynyloxy, lower alkynylamino, cyclo(lower)alkyl, cyclo(lower)alkyloxy, cyclo(lower)alkylamino, aryl, aryloxy, arylamino, a heterocyclic group or amino substituted with a heterocyclic group, each of which may be substituted with suitable substituent(s); or acyl;
Z is a single bond, —CO— or —SO₂—,
E is lower alkylene optionally substituted with suitable substituent(s),
X is CH or N,
J is a single bond, lower alkylene or 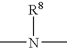
wherein R⁸ is hydrogen, lower alkyl, substituted-lower alkyl, an N-protective group, aryl, acyl or a heterocyclic group,
Q is —CH₂—, —CO—, —SO₂— or —N═CH—, and
R⁵ and R⁶ are each hydrogen or lower alkyl, or are taken together to form lower alkylene optionally condensed with a cyclic hydrocarbon or a heterocyclic ring, provided that when X is N, then
1) J is a single bond, and Q is —CH₂—, —CO— or —SO₂—, or
2) J is lower alkylene,
or pharmaceutically acceptable salts thereof.

16. The use of a compound having α7 nicotinic acetylcholine receptor activation property according to claim 13, wherein the compound has the following formula [II-2]: 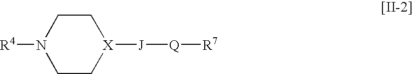
wherein
R⁴ is acyl,
R⁷ is aryl, aryloxy or arylamino, the aryl moiety of all of which may be substituted with halogen; pyridyl; or pyridylamino;
X is CH or N,
J is a single bond, lower alkylene or 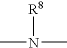
wherein R⁸ is hydrogen, lower alkyl or an N-protective group,
Q is —CH₂—, —CO— or —SO₂—, provided that when X is N, then J is a single bond or lower alkylene, or pharmaceutically acceptable salts thereof.

17. The use of a compound having α7 nicotinic acetylcholine receptor activation property according to any of claim 13 to claim 16, which is for the production of an agent for the prophylaxis and/or treatment of cerebral diseases.

18. The use of a compound having α7 nicotinic acetylcholine receptor activation property according to claim 17, which is for the production of an agent for the prophylaxis and/or treatment of dementia or amnesia.

19. A pharmaceutical composition for modulating excitatory synaptic transmission, which comprises a compound having α7 nicotinic acetylcholine receptor activation property, and a pharmaceutically acceptable carrier or excipient.

20. The pharmaceutical composition for modulating excitatory synaptic transmission of claim 19, wherein the compound has the following formula [I]: 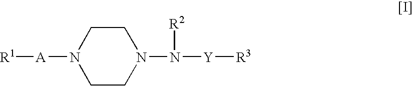
wherein
R¹ is lower alkyl, aryl, ar(lower)alkoxy or heterocyclic group, each of which may be substituted with halogen,
R² is hydrogen atom or lower alkyl,
R³ is cyclo(lower)alkyl, aryl or ar(lower)alkyl, each of which may be substituted with halogen,
A is —CO—, —SO₂— or lower alkylene, and
Y is —CO—, —SO₂— or —CONH— or pharmaceutically acceptable salts thereof.

21. The pharmaceutical composition for modulating excitatory synaptic transmission of claim 19, wherein the compound has the following formula [II-1]: 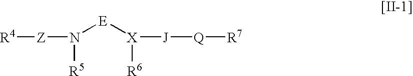
wherein
R⁴ is acyl,
R⁷ is lower alkyl, lower alkoxy, lower alkylamino, lower alkenyl, lower alkenyloxy, lower alkenylamino, lower alkynyl, lower alkynyloxy, lower alkynylamino, cyclo(lower)alkyl, cyclo(lower)alkyloxy, cyclo(lower)alkylamino, aryl, aryloxy, arylamino, a heterocyclic group or amino substituted with a heterocyclic group, each of which may be substituted with suitable substituent(s); or acyl;
Z is a single bond, —CO— or —SO₂—,
E is lower alkylene optionally substituted with suitable substituent(s),
X is CH or N,
J is a single bond, lower alkylene or 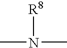
wherein R⁸ is hydrogen, lower alkyl, substituted-lower alkyl, an N-protective group, aryl, acyl or a heterocyclic group,
Q is —CH₂—, —CO—, —SO₂— or —N═CH—, and
R⁵ and R⁶ are each hydrogen or lower alkyl, or are taken together to form lower alkylene optionally condensed with a cyclic hydrocarbon or a heterocyclic ring, provided that when X is N, then
1) J is a single bond, and Q is —CH₂—, —CO— or —SO₂—, or
2) J is lower alkylene,
or pharmaceutically acceptable salts thereof.

22. The pharmaceutical composition for modulating excitatory synaptic transmission of claim 19, wherein the compound has the following formula [II-2]: 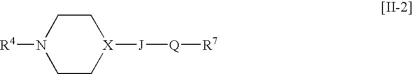
wherein
R⁴ is acyl,
R⁷ is aryl, aryloxy or arylamino, the aryl moiety of all of which may be substituted with halogen; pyridyl; or pyridylamino;
X is CH or N,
J is a single bond, lower alkylene or 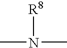
wherein R⁸ is hydrogen, lower alkyl or an N-protective group,
Q is —CH₂—, —CO— or —SO₂—, provided that when X is N, then J is a single bond or lower alkylene, or pharmaceutically acceptable salts thereof.

23. The pharmaceutical composition for modulating excitatory synaptic transmission of any of claim 19 to claim 22, which is a pharmaceutical composition for the prophylaxis or treatment of cerebral diseases.

24. The pharmaceutical composition for modulating excitatory synaptic transmission of claim 23, which is a pharmaceutical composition for the prophylaxis or treatment of dementia or amnesia.

25. A method for screening an agent for modulating excitatory synaptic transmission, which comprises using α7 nicotinic acetylcholine receptor activation property as an index.

26. The screening method of claim 25, which is a screening method of an anti-dementia agent or anti-amnesia agent.

27. The agent for modulating excitatory synaptic transmission of claim 1, wherein the compound having α7 nicotinic acetylcholine receptor activation property is a compound obtained by the screening method of any of claim 25 to claim 26.

28. The method for modulating excitatory synaptic transmission according to claim 7, wherein the compound having α7 nicotinic acetylcholine receptor activation property is a compound obtained by the screening method of any of claim 25 to claim 26.

29. The use of a compound having α7 nicotinic acetylcholine receptor activation property according to claim 13, wherein the compound having α7 nicotinic acetylcholine receptor activation property is obtained by the screening method of any of claim 25 to claim 26.

30. The pharmaceutical composition for modulating excitatory synaptic transmission of claim 19, wherein the compound having α7 nicotinic acetylcholine receptor activation property is a compound obtained by the screening method of any of claim 25 to claim 26.

31. A compound selected by the screening method described in any of claim 25 to claim 26.
